(19) United States
(12) Patent Application Publication
(10) Pub. No.: US 2018/0263919 A1
(43) Pub. Date: Sep. 20, 2018

(54) THERAPEUTIC ELECTROSPUN FIBER COMPOSITIONS

(71) Applicant: The Johns Hopkins University, Baltimore, MD (US)

(72) Inventors: Ahmet Hoke, Towson, MD (US); Sing Y. Chew, Baltimore, MD (US); Ruifa Mi, Baltimore, MD (US); Kam W. Leong, Durham, NC (US)

(21) Appl. No.: 15/916,749

(22) Filed: Mar. 9, 2018

Related U.S. Application Data
(63) Continuation of application No. 15/498,075, filed on Apr. 26, 2017, now abandoned.
Application No. 15/498,075 is a continuation of application No. 14/605,585, filed on Jan. 26, 2015, now abandoned.
Application No. 14/605,585 is a continuation of application No. 14/207,830, filed on Mar. 13, 2014, now abandoned.
Application No. 14/207,830 is a continuation of application No. 12/223,571, filed on Nov. 12, 2009, now abandoned.
(60) Provisional application No. 60/765,069, filed on Feb. 2, 2006.

(51) Int. Cl. A61K 9/70; A61L 27/18; A61K 31/7088; A61B 17/11; A61L 31/04; A61L 27/54; A61L 27/44; A61B 17/00

(52) U.S. Cl. CPC A61K 9/70; A61L 27/18; A61K 31/7088; A61B 17/1128; A61L 2300/414; A61L 27/54; A61L 27/44; A61B 2017/00526; A61B 2017/00004; A61L 31/047

(57) ABSTRACT

The instant invention provides electrospun fiber compositions comprising one or more polymers and one or more biologically active agents. In specific embodiments, the biologically active agents are nerve growth factors. In certain embodiments, the electrospun fiber compositions comprising one or more biologically active agents are on the surface of a film, or a tube. The tubes comprising the electrospun fiber compositions of the invention can be used, for example, as nerve guide conduits.

### RELATED APPLICATIONS

This application is a continuation application of U.S. Ser. No. 14/605,585, filed Jan. 26, 2015, which is a continuation application of U.S. Ser. No. 14/207,830, filed Mar. 13, 2014, which is a continuation application of U.S. Ser. No. 12/223,571, filed Nov. 12, 2009, which is a national stage application filed under 35 U.S.C. § 371 of International Application No. PCT/US2006/017444, filed May 4, 2006, which claims the benefit of priority under 35 U.S.C. § 119(e) to U.S. Provisional Application No. 60/765,069 filed Feb. 2, 2006, each of which is incorporated herein by reference in its entirety.

### GOVERNMENT SUPPORT

This invention was made with government support under Grant No. EB003447 awarded by National Institutes of Health. The government has certain rights in the invention.

### BACKGROUND OF THE INVENTION

There is a need in the art for improved compositions that release therapeutics, e.g., biological therapeutics, in a biologically active form over a prolonged period of time. For example, processes such as nerve regenereation would benefit from such a composition. Peripheral nerve regeneration and functional recovery is often ineffective over long lesion gaps despite surgical interventions and entubulation of the injured nerve. By far, the most common and efficient method of treatment is the use of autografts for long lesion gaps. However, drawbacks such as requirement of a second surgery, lack of available donor nerves, loss of donor nerve function, neuroma formation, and unacceptable scarring (Wang, Cai et al. 2002; Francel, Smith et al. 2003; Bunting, Silvio et al. 2005) justify the continuing search for better alternatives. The use of empty synthetic nerve guides has been one of the popular choices. These synthetic tubes, however, are only successful in bridging short nerve gaps such as 10 mm in the rat model (Ceballos, Navarro et al. 1999; Arai, Lundborg et al. 2000; Wang, Cai et al. 2002; Ngo, Waggoner et al. 2003; Cai, Peng et al. 2004). Additionally, there appears to be a species-dependent critical defect gap size, e.g. 15 mm in rats, beyond which the regeneration of injured nerves seldom occurs in these empty synthetic nerve guides (Ceballos, Navarro et al. 1999; Francel, Smith et al. 2003; Udina, Rodriguez et al. 2004).

The lack of regeneration of injured nerves across large lesion gaps may be partially due to inadequate formation of the extracellular matrix during the initial phase of recovery to provide a scaffold on which cells migrate and proliferate from the proximal to the distal end of the nerve (Ceballos, Navarro et al. 1999). As a result, different approaches have been taken to encourage nerve regeneration over long lesion gaps in synthetic nerve guides. To provide a scaffold for cell attachment (Ceballos, Navarro et al. 1999; Ngo, Waggoner et al. 2003), pre-filling of synthetic nerve conduits with dialyzed plasma, collagen or laminin-containing gels have been adopted (Ceballos, Navarro et al. 1999; Ngo, Waggoner et al. 2003). These fillings can effectively improve nerve regeneration (Ceballos, Navarro et al. 1999; Ngo, Waggoner et al. 2003). The introduction of contact guidance to accelerate tissue regeneration using aligned structures such as microfilaments (20 to 100 mm in diameter) (Dahlin and Lundborg 1999; Arai, Lundborg et al. 2000; Rangappa, Romero et al. 2000; Ngo, Waggoner et al. 2003; Cai, Peng et al. 2004; Yoshii, Shima et al. 2004; Bunting, Silvio et al. 2005), micropatterns (Rutkowski, Miller et al. 2004) and aligned collagen gel (Ceballos, Navarro et al. 1999) is another alternative. While nerve guides provide a general direction for regenerating nerves, aligned inclusions provide the local contact guidance leading to directional axonal outgrowth towards the distal stump of the nerve (Cai, Peng et al. 2004), which may be crucial for nerve regeneration (Ceballos, Navarro et al. 1999; Rutkowski, Miller et al. 2004). Both in vitro and in vivo observations support the potential of aligned topographies in enhancing nerve regeneration. In vitro experiments illustrate that neurites respond to surface topographies by extending and growing along the length of microgrooves on flat substrates (Rutkowski, Miller et al. 2004), and microfilaments could direct Schwann cell migration and growth longitudinally (Ngo, Waggoner et al. 2003). In vivo studies demonstrate enhanced sciatic nerve regeneration through morphometric analyses over lesion gaps of 5-20 mm in rats (Dahlin and Lundborg 1999; Arai, Lundborg et al. 2000; Ngo, Waggoner et al. 2003; Cai, Peng et al. 2004; Yoshii, Shima et al. 2004; Bunting, Silvio et al. 2005) and 6 mm in mice (Ceballos, Navarro et al. 1999).

Accordingly, the need exists for new methods and compositions for administering therapeutic molecules over prolonged periods of time.

### SUMMARY OF THE INVENTION

We have discovered that electrospun fiber compositions comprising one or more therapeutic agents are effective for releasing therapeutic agents over prolonged periods of time. In particular, we have now shown that polymeric electrospun fiber compositions comprising biological therapeutics effectively release active biological molecules for prolonged periods of time. We also have shown that nerve guide conduits comprising an electrospun fiber composition comprising a therapeutic agents are effective for stimulating nerve growth.

Accordingly, in one aspect, the instant invention provides electrospun fiber compositions comprising one or more polymers and one or more biological therapeutics.

In one embodiment, the electrospun fiber compositions comprise one or more of the following therapeutics: a polypeptide, polypeptide fragment, nucleic acid molecule, or a carbohydrate. In a specific embodiment, the biological therapeutics are one or more polypeptides, e.g., a growth factor, chemokine, cytokine, receptor, antibody, scFv, antibody fragment or a combination thereof.

In a related embodiment, the electrospun fiber compositions, further comprise additional polypeptides, e.g., filler polypeptides. These filler polypeptides can be, for example, albumins such as human serum albumin.

In one embodiment, the electrospun fiber compositions comprise randomly oriented fibers. In alternative embodiments, the compositions comprise aligned fibers. The electrospun fiber compositions can be produced by electrospinning methods known in the art, e.g., uniaxial electrospinning, coaxial electrospinning or multiaxial electrospinning.

In another embodiment, the electrospun fiber compositions have an average fiber diameter between about 10 nm and 10 um. In particular embodiments, the average fiber diameter is between about 100 nm and 1 um.

In another embodiment, the electrospun fiber compositions, comprise one or more polymers, e.g., synthetic polymers, natural polymers, protein engineered biopolymers or combinations thereof.

In exemplary embodiments, the electrospun fiber compositions comprise a polyester or derivative thereof. In particular embodiments, the polyester is a poly(phosphoester) polymer, e.g., poly (ε-caprolactore-co-ethyl ethylene phosphate (PCLEEP).

In another embodiment, the electrospun fiber compositions, comprises at least about 5% biological therapeutic by weight. In another embodiment, the composition comprises at least 10% biological therapeutic by weight.

In one embodiment, the electrospun fiber compositions are biodegradable. In an alternative embodiment, the compositions are non-biodegradable.

In another embodiment, the electrospun fiber compositions releases biologically active therapeutic molecules for at least about 2 months.

In another embodiment, the electrospun fiber compositions, are on a film, e.g., metal, ceramics or polymer films. In a preferred embodiment, the film is a polymer film, e.g., a PCLEEP film of aligned fibers. In a related embodiment, the film is formed into a tube and the electrospun fiber composition is on a surface of the tube, e.g., the inner surface of the tube.

In another aspect, the invention provides an electrospun fiber composition comprising PCLEEP and one or more therapeutically active molecules. Exemplary therapeutically active molecules include, but are not limited to, a polypeptide, polypeptide fragment, nucleic acid molecule, small molecule, ribozyme, shRNA, RNAi, antibody, antibody fragment, scFv, carbohydrate, or combinations thereof. In a specific embodiment, the therapeutically active molecule is a small molecule. In one exemplary embodiment, the small molecule is retinoic acid.

In one embodiment, the electrospun fiber compositions comprise randomly oriented fibers. In alternative embodiments, the compositions comprise aligned fibers. The electrospun fiber compositions can be produced by electrospinning methods known in the art, e.g., uniaxial electrospinning, coaxial electrospinning or multiaxial electrospinning.

In another embodiment, the electrospun fiber compositions, comprises at least about 5% biological therapeutic by weight. In another embodiment, the composition comprises at least 10% biological therapeutic by weight.

In another embodiment, the therapeutically active molecule is encapsulated, e.g., encapsulated in chromium.

In another embodiment, the electrospun fiber compositions releases biologically active therapeutic molecules for at least about 2 months.

In another embodiment, the electrospun fiber compositions, are on a film, e.g., metal, ceramics or polymer films. In a preferred embodiment, the film is a polymer film, e.g., a PCLEEP film of aligned fibers. In a related embodiment, the film is formed into a tube and the electrospun fiber composition is on a surface of the tube, e.g., the inner surface of the tube.

In another aspect, the instant invention provides a cylindrical polymer film comprising an inner surface, an outer surface, and a lumen, wherein an electrospun fiber composition comprising one or more polymers and one or more therapeutically active molecules is present on the inner or outer surface of the cylindrical polymer film.

In one embodiment, the electrospun fiber composition is on the inner surface.

In one embodiment, the electrospun fiber compositions comprise randomly oriented fibers. In alternative embodiments, the compositions comprise aligned fibers. The electrospun fiber compositions can be produced by electrospinning methods known in the art, e.g., uniaxial electrospinning, coaxial electrospinning or multiaxial electrospinning.

In another embodiment, the electrospun fiber compositions, comprises at least about 5% biological therapeutic by weight. In another embodiment, the composition comprises at least 10% biological therapeutic by weight.

In one embodiment, the one or more therapeutically active molecules are one or more polypeptides. In a related embodiment, the one or more polypeptides comprise a growth factor, e.g., NGF or GNDF.

In another embodiment, the one or more polymers comprise a synthetic polymer, a natural polymer, a protein engineered biopolymer or a combination thereof. In a specific embodiment, the one or more polymers comprise a polyester or derivative thereof. In a further specific embodiment, the polyester is a poly (phosphoester), e.g., poly (ε-caprolactore-co-ethyl ethylene phosphate (PCLEEP).

In one embodiment, the polymers are biodegradable. In an alternative embodiment, the polymers are non-biodegradable.

In another aspect, the instant invention provides a nerve guide conduit comprising a cylindrical polymer film comprising on the interior surface an electrospun fiber composition comprising one or more polymers and one or more therapeutically active molecules that induce nerve growth.

In one embodiment, the electrospun fiber compositions comprise randomly oriented fibers. In alternative embodiments, the compositions comprise aligned fibers. The electrospun fiber compositions can be produced by electrospinning methods known in the art, e.g., uniaxial electrospinning, coaxial electrospinning or multiaxial electrospinning.

In another embodiment, the electrospun fiber compositions, comprises at least about 5% biological therapeutic by weight. In another embodiment, the composition comprises at least 10% biological therapeutic by weight.

In another embodiment, the composition releases biologically active therapeutic molecules for at least about 2 months.

In one embodiment, the polymers are biodegradable. In an alternative embodiment, the polymers are non-biodegradable.

In another embodiment, the instant invention provides a polymer film comprising on one surface an electrospun fiber composition comprising one or more polymers and one or more biologically active molecules.

In one embodiment, the film is therapeutic. In another embodiment, the one or more biologically active molecules are therapeutic. In a related embodiment, the one or more biologically active molecules are selected from the group consisting of a small molecule, polypeptide, polypeptide fragment, nucleic acid molecule, carbohydrates, and combinations thereof.

In a specific embodiment, the one or more biologically active molecules comprise a small molecule. In an exemplary embodiment, the small molecule is retinoic acid.

In another specific embodiment, the one or more biologically active molecules comprise a polypeptide. In a related embodiment, the polypeptide is a growth factor, e.g., NGF or GNDF.

In one embodiment, the electrospun fibers are randomly oriented fibers. In another embodiment, the electrospun fiber is an aligned fiber.

In one embodiment, the electrospun fiber compositions comprise randomly oriented fibers. In alternative embodiments, the compositions comprise aligned fibers. The electrospun fiber compositions can be produced by electrospinning methods known in the art, e.g., uniaxial electrospinning, coaxial electrospinning or multiaxial electrospinning.

In another embodiment, the one or more polymers comprise a synthetic polymer, a natural polymer, a protein engineered biopolymer or a combination thereof. In one embodiment, the one or more polymers comprise a polyester or derivative thereof. In a specific embodiment, the polyester is a poly(phosphoester), e.g., poly (ε-caprolactore-co-ethyl ethylene phosphate (PCLEEP).

In another embodiment, the electrospun fiber compositions, comprises at least about 5% biological therapeutic by weight. In another embodiment, the composition comprises at least 10% biological therapeutic by weight.

In another embodiment, the composition releases biologically active therapeutic molecules for at least about 2 months.

In one embodiment, the polymers are biodegradable. In an alternative embodiment, the polymers are non-biodegradable.

In a related method, the film is a substrate for cell growth.

### DESCRIPTION OF THE DRAWINGS

FIGS. 1A-1D depict: (FIG. 1A) aligned PCLEEP fibers without proteins, electrospun at 4.5 ml/h. Fiber diameter, ϕ=5.01±0.24 μm; (FIG. 1B) aligned BSA encapsulated PCLEEP fibers electrospun at 4.5 ml/h, ϕ=2.80±0.15 μm; (FIG. 1C) size distribution of plain PCLEEP fibers; and (FIG. 1D) Aligned BSA encapsulated PCLEEP fibers electrospun at 1 ml/h, ϕ=0.46±0.027 μm. Fiber diameter expressed as mean±S.E.

FIGS. 2A-2B depict: (FIG. 2A) the percentage mass loss of aligned PCLEEP fibers without proteins versus degradation time, n=3, mean±S.E. Significant mass loss was observed, P<0.01, paired sample t-test; and (FIG. 2B) Molecular weight change versus time of PCLEEP fibers and film and PCL film.

FIGS. 3A-3B depict PLCEEP fibers incubated in PBS at 37° C. after 57 days (FIG. 3A) 500× magnification; and (FIG. 3B) 5000× magnification.

FIG. 4 depicts swelling behavior of PCLEEP sheets incubated in distilled water at 37° C. n=4, mean±S.E.

FIGS. 5A-5B depict FITC-BSA-encapsulated PCLEEP electrospun fibers.

FIG. 6 depicts the release profile of NGF from PCLEEP electrospun fibers. Comparison of first 60% of NGF release profile with Fickian diffusion from monodispersed cylinders (dotted line). n=3, mean±S.E.

FIGS. 7A-7D depict: (FIG. 7A) positive control of PC12 cells in NGF; (FIG. 7B) negative control of PC12 cells in plain serum-free RPMI medium; (FIG. 7C) PC12 cells in Day 1 supernatant; and (FIG. 7D) PC12 cells in Day 85 supernatant.

FIG. 8 depicts the percentage of PC12 cells showing signs of differentiation (♦), and mass of NGF added to each culture well (◯) at various time points. n=650, mean±S.E.

FIGS. 9A-9C depict a schematic of the production of a nerve guide conduit. FIG. 9A depicts the synthesis of PCLEEP. FIG. 9B depicts the fabrication of nerve conduits. FIG. 9C shows the in vitro cumulative release profile of GDNF from aligned GDNF-encapsulated PCLEEP electrospun fibers incubated at 37° C. under static conditions for 3 months (n=4, mean±SE).

FIGS. 10A-10D depict cross-sectional views of nerve conduits with nerve wires: FIG. 10A) NW-L; FIG. 10B) NW-CL; inset: higher magnification views of cross-sections; FIG. 10C) aligned PCLEEP fibers in nerve guide conduits, GDNF-encapsulated fiber diameter, φ, =3.96±0.14 μm and plain PCLEEP fiber φ=5.08±0.05 μm; and FIG. 10D) inner surface of empty nerve guide conduit.

FIGS. 11A-11D depict light micrographs of the cross-sections of regenerated sciatic nerves, 8-10 mm from the proximal end of a control (FIG. 11A), NW-L (no GDNF), dashed circles indicate nerve wires (FIG. 11B); NW-CL (no GDNF), dashed circle indicate nerve wire (FIG. 11C), and NW-L (with GDNF) (FIG. 11D).

FIG. 12A and FIG. 12B depict the total number of myelinated axons at 8-10 mm from the proximal end of each regenerated sciatic nerve, *p<0.05, **p<0.01(FIG. 12A); and the cross-sectional area of regenerated nerve at 8-10 mm from proximal end, *p<0.05 and ** p<0.01 (FIG. 12B).

FIG. 13 depicts the G ratio of the nerves. The G ratio is defined as ratio of diameter of axon to the diameter of the entire myelinated fiber.

FIGS. 14A-14C depict TEM micrographs of cross-sections of regenerated sciatic nerve, 8-10 mm from the proximal end of control, showing the absence of myelinated axons and the presence of fibrous tissues (FIG. 14A); NW-L (no GDNF), showing the tendency of myelinated axons regenerating in close proximity to PCLEEP nerve wires (circled) (FIG. 14B); and NW-L (with GDNF), demonstrating the presence of a large number of myelinated axons (FIG. 14C).

FIGS. 15A-15B depict immunofluorescent micrographs of the cross-section of a regenerated sciatic nerve 5-8 mm from proximal end. Activated macrophages found mostly along the periphery of the sciatic nerve. Green: ED1; blue: DAPI(FIG. 15A); and light micrograph of the cross-section of a regenerated sciatic nerve from NW-L (no GDNF), 5-8 mm from proximal end, under H&E staining. No acute immune response was observed. Dashed circles: PCLEEP nerve wires (FIG. 15B).

FIGS. 16A-16C depict the percentage of rats per group that showed functional recovery, * p<0.1, Fisher-Irwin test (FIG. 16A); CMAP amplitude (FIG. 16B); and CMAP latency (FIG. 16C). NWCL(no GDNF): n=2, NW-L (no GDNF): n=3, NW-L (GDNF): n=4.

### DETAILED DESCRIPTION OF THE INVENTION

The instant invention provides compositions, e.g., electrospun fiber compositions, comprising a therapeutic agent and an electrospun matrix, e.g., a polymer matrix. The inventors of the instant technology have found that the compositions described herein can release biologically active therapeutic agents over a prolonged period of time.

The following definitions will be useful in understanding the instant invention.

As used herein, the term “therapeutically active molecules” includes a “drug” and means a molecule, group of molecules, complex or substance administered to an organism for diagnostic, therapeutic, preventative medical, or veterinary purposes. This term include externally and internally administered topical, localized and systemic human and animal pharmaceuticals, treatments, remedies, nutraceuticals, cosmeceuticals, biologicals, devices, diagnostics and contraceptives, including preparations useful in clinical screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes. Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a growth factor, e.g., NGF or GNDF, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an anxiolytic agent, a peptidic agent, a biopolymeric agent, an antineoplastic agent, a laxative, an antidiarrheal agent, an antimicrobial agent, an antifingal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

As used herein, the term “biological therapeutic” is intended to mean a subset of therapeutically active molecules that are a polypeptide or nucleic acid molecule. In specific embodiments, the biological therapeutic is an agent that induces or enhances nerve growth, i.e., a neurotrophic agent. Examples of useful neurotrophic agents are αFGF (acidic fibroblast growth factor), FGF (basic FGF), NGF (nerve growth factor), BDNF (brain derived neurotrophic factor), CNTF (ciliary neurotrophic factor), MNGF (motor nerve growth factor), NT-3 (neurotrophin-3), GDNF (glial cell line-derived neurotrophic factor), NT4/5 (neurotrophin4/5), CM101, HSP-27 (heat shock protein-27), IGF-I (insulin-like growth factor), IGF-II (insulin-like growth factor 2), PDGF (platelet derived growth factor) including PDGF-BB and PDGF-AB, ARIA (acetylcholine receptor inducing activity), LIF (leukemia inhibitory factor), VIP (vasoactive intestinal peptide), GGF (glial growth factor), and IL-1 (interleukin-1). In a preferred embodiment, the biological therapeutic is NGF or GNDF.

As used herein, the term “electrospinning” is intended to mean a process that uses an electric field to draw a solution comprising, for example, a polymer or a ceramic from the tip of the capillary to a collector. A high voltage DC current is applied to the solution which causes a jet of the solution to be drawn towards the grounded collector screen. Once ejected out of the capillary orifice, the charged solution jet gets evaporated to form fibers and the fibers get collected on the collector. The size and morphology of the fibers thus obtained depends on a variety of factors such as viscosity of the solution, molecular weight, nature of the polymer or ceramic and other parameters regarding the electrospinning apparatus. The electrospinning process to form polymer nanofibers has been demonstrated using a variety of polymers [Huang, et al. Composites Science and Technology 2003; 63]. Exemplary polymers used in electrospinning methods of the invention include those disclosed in U.S. Pat. No. 6,852,709, issued Feb. 8, 2005. Electrostatic spinning is a process by which polymer fibers of nanometer to micrometer size in diameters and lengths up to several kilometers can be produced using an electrostatically driven jet of polymer solution or polymer melt. The polymer solution or melt may comprise one or more therapeutically active molecules at concentrations determined by the ordinary skilled artisan.

The term “treated,” “treating” or “treatment” includes the diminishment or alleviation of at least one symptom associated or caused by the state, disorder or disease being treated. Moreover, treatment includes the partial or complete regeneration of nerve fibers in a subject.

The term “subject” is intended to include organisms needing treatment. Examples of subjects include mammals, e.g., humans, dogs, cows, horses, pigs, sheep, goats, cats, mice, rabbits, rats, and transgenic non-human animals. In certain embodiments, the subject is a human.

As used herein, the term “uniaxial electrospinning” is intended to mean the electrospinning of a single electrospinning solution supply that is dispensed from a single spinneret.

As used herein, the term “coaxial electrospinning” is intended to mean the electrospinning of a single electrospinning solution supply that comprises of two different solutions that are physically separated from each other and that are dispensed from two separate spinnerets that share the same axis of symmetry.

As used herein, the term “multiaxial electrospinning” is intended to mean the electrospinning of a single electrospinning solution supply that comprises of multiple solutions that are physically separated from each other and that are dispensed through multiple spinnerets that share the same axis of symmetry.

As used herein, the term “filler polypeptide” is intended to mean one or more polypeptides that are used in the electrospun fiber compositions for reasons other than a therapeutic effect. For example, these filler polypeptides may be polypeptides used to stabilize a biological therapeutic, e.g., extend the length of time that biological therapeutic molecules maintain their activity. Polypeptides useful as stabilizer polypeptides include polypeptides that will not elicit an immune response in the subject to which the composition will be administered. For example, human serum albumin is a suitable filler polypeptide for use in compositions designed for use or administration to human beings.

As used herein, the term “polymer” generally includes, but is not limited to, homopolymers, copolymers, such as for example, block, graft, random and alternating copolymers, terpolymers, etc., and modifications thereof. In addition, unless otherwise specifically limited, the term “polymer” also includes all possible geometric configurations of the molecule. In specific embodiments, the polymers used in the compositions of the invention are polyesters. An exemplary polyester used in the compositions of the invention is PCLEEP.

As used herein, the term “poly (ε-caprolactore-co-ethyl ethylene phosphate (PCLEEP)” is intended to mean a polymer described in U.S. Pat. No. 6,852,709 having the following structure:

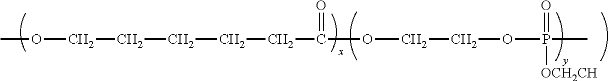

As used herein, the term “tube” is intended to mean composition of matter having an interior surface, and exterior surface, a lumen and openings on the two ends. The tubes of the invention may be made by from the a film by rolling the film and joining the film where it overlaps. Tubes of the invention can be made of, for example, plastics, polymers, ceramics or metals.

As used herein, “biocompatible” means the ability of an object to be accepted by and to function in a recipient without eliciting a significant foreign body response (such as, for example, an immune, inflammatory, thrombogenic, or the like response). For example, when used with reference to one or more of the polymeric materials of the invention, biocompatible refers to the ability of the polymeric material (or polymeric materials) to be accepted by and to function in its intended manner in a recipient.

As used herein, “therapeutically effective amount” refers to that amount of a therapeutic agent alone that produces the desired effect (such as treatment of a medical condition such as a disease or the like, or alleviation of a symptom such as pain) in a patient. In some aspects, the phrase refers to an amount of therapeutic agent that, when incorporated into a composition of the invention, provides a preventative effect sufficient to prevent or protect an individual from future medical risk associated with a particular disease or disorder. A physician or veterinarian of ordinary skill can readily determine and prescribe the effective amount of the bioactive agent required to treat and/or prevent the progress of the condition.

The electrospun fiber compositions of the invention are made from any of a number of materials that are suitable for electrospinning. Specifically, the compositions of the invention comprise polymers or ceramics. In preferred embodiments, the electrospun fiber compositions of the invention are made of polymers. In exemplary embodiments, the polymers used to make the compositions of the invention are polyesters, e.g., PCLEEP.

Compositions of the Invention

The instant invention provides electrospun fiber compositions comprising one or more therapeutic agents and one or more agents suitable for electrospinning, e.g., polymers or ceramics. In preferred embodiments the electrospun fiber compositions comprise polymers, e.g., polyesters or poly (phosphoesters).

In one embodiment, the compositions comprise a electrospun fiber composition comprising one or more therapeutic agents. In certain embodiments, the composition can be encapsulated in materials known to one of skill in the art to control the rate of degradation of the composition and ultimately the rate of release of the therapeutic agents.

Additionally, the density of the electrospun fiber composition can be adjusted by the ordinary skilled artisan to increase or decrease the length of time that therapeutic molecules are released from the composition. Moreover, varying the density of the electrospun fiber composition can be used to modulate the amount of the therapeutic that is released per unit of time.

The compositions can be comprised of aligned or randomly oriented fibers. Moreover, the compositions can be produced by electrospinning methods that are known in the art. For example, the compositions can be produced by uniaxial, coaxial or multiaxial electrospinning.

The average fiber diameter of the electrospun fibers in the compositions of the invention can be, for example, from about 10 nm to about 100 μm. In further exemplary embodiments, the average size of the electrospun fibers is between about 50 nm and about 50 μm, between about 10 nm and about 10 μm or between about 100 nm and about 1 μm.

The compositions of the invention may also be on the surface of a film or tube, e.g., a nerve guide conduit. In exemplary embodiments, the electrospun fiber composition comprises an electrospun fiber composition comprising one or more therapeutic agents on the interior surface of a tube. This tube is useful as a nerve guide conduit to aid in nerve regeneration. As set forth herein, the nerve guide conduits of the invention may comprise one or more neuropathic compositions, e.g., biological molecules that stimulate the growth of nerve cells, for example, NGF or GNDF.

In other embodiments, the electrospun fiber composition comprising one or more therapeutic agents is produced on the surface of a film which can be applied to a specific area of a subject in need of treatment. For example, the compositions can comprise growth factors that stimulate the growth of, for example, cardiac cells, epithelial cells, liver cells, or bladder cells. In further exemplary embodiments, the electrospun fiber compositions deposited on the surface of a film act as growth a growth substrate for stem cells by incorporating the necessary factors into the composition. Moreover, factors that result in the differentiation of stem cells can be incorporated into the composition resulting in a differentiation of stem cells for therapeutic or research applications.

In certain embodiments of the invention, the electrospun fiber compositions of the invention comprise one or more therapeutic molecules. The therapeutic molecules may comprise about 0.01, 0.05, 0.1, 0.5, 1, 2, 3, 4, 5, 6, 7, 8, 9, 10, 11, 12, 13, 14, or 15% of the composition by weight. In preferred embodiments, the therapeutic comprises about 1-10% of the electrospun fiber composition by weight. In another embodiments, the therapeutics comprise about 1-5% of the electrospun fiber composition by weight.

In other embodiments, the electrospun fiber compositions of the invention comprise one or more biological therapeutics. The instant invention provides electrospun fiber compositions that release biologically active biological therapeutics for prolonged periods of time. For example, the electrospun fiber compositions of the invention release biologically active therapeutics for periods of 1 day to 18 months. Specifically, the electrospun fiber compositions of the invention release biologically active therapeutics for at least about 1 day, 2 days, 3 days, 4 days, 5 days, 15 days, 30 days, 45 days, 60 days, 90 days, 120 days, 180 days, 360 days, or more. In preferred embodiments, the electrospun fiber compositions of the invention release biologically active therapeutics for about 30 to about 120 days.

The electrospun fiber compositions of the invention are effective as time release formulation for the delivery of a therapeutic agent to a subject in need thereof over a prolonged period of time.

Exemplary therapeutically active agents include, for example, biological agents and small molecules. For example, therapeutically active agents include, but are not limited to, neuropathic agents; thrombin inhibitors; antithrombogenic agents; thrombolytic agents (such as plasminogen activator, or TPA: and streptokinase); fibrinolytic agents; vasospasm inhibitors; calcium channel blockers; vasodilators; antihypertensive agents; clotting cascade factors (for example, protein S); anticoagulant compounds (for example, heparin and nadroparin, or low molecular weight heparin); retinoic acid; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, minocycline, doxycycline, vancomycin, kanamycin, cephalosporins such as cephalothin, cephapirin, cefazolin, cephalexin, cephardine, cefadroxil, cefamandole, cefoxitin, cefaclor, cefuroxime, cefonicid, ceforanide, cefitaxime, moxalactam, cetizoxime, ceftriaxone, cefoperazone), geldanamycin and analogues, antifungals (such as amphotericin B and miconazole), and antivirals (such as idoxuridine trifluorothymidine, acyclovir, gancyclovir, interferon, α methyl-P-adamantane methylamine, hydroxy-ethoxymethyl-guanine, adamantanamine, 5-iodo-deoxyuridine, trifluorothymidine, interferon, adenine arabinoside); inhibitors of surface glycoprotein receptors; antiplatelet agents (for example, ticlopidine); antimitotics; microtubule inhibitors; anti-secretory agents; active inhibitors; remodeling inhibitors; antisense nucleotides (such as morpholino phosphorodiamidate oligomer); anti-metabolites; antiproliferatives (including antiangiogenesis agents, taxol, sirolimus (rapamycin), analogues of rapamycin (“rapalogs”), tacrolimus, ABT-578 from Abbott, everolimus, paclitaxel, taxane, vinorelbine); anticancer chemotherapeutic agents; anti-inflammatories; non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); mydriatics (such as atropine, cyclopentolate, homatropine, scopolamine, tropicamide, eucatropine, hydroxyamphetamine); sympathomimetics (such as epinephrine); antineoplastics (such as carmustine, cisplatin, fluorouracil); immunological drugs (such as vaccines and immune stimulants); hormonal agents (such as estrogens, estradiol, progesterol, progesterone, insulin, calcitonin, parathyroid hormone, peptide and vasopressin hypothalamus releasing factor); beta adrenergic blockers (such as timolol maleate, levobunolol HCl, betaxolol HCl); immunosuppressive agents, growth hormone antagonists, growth factors (such as epidermal growth factor, fibroblast growth factor, platelet derived growth factor, transforming growth factor beta, somatotropin, fibronectin, insulin-like growth factor (IGF)); carbonic anhydrase inhibitors (such as dichlorophenamide, acetazolamide, methazolamide); inhibitors of angiogenesis (such as angiostatin, anecortave acetate, thrombospondin, anti-VEGF antibody such as anti-VEGF fragment—ranibizumab (Lucentis)); dopamine agonists; radiotherapeutic agents; peptides; proteins; enzymes; nucleic acids and nucleic acid fragments; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and the like.

In one embodiment, the anti-inflammatory is a nonsteroidal antiinflammatory drug (NSAID) that inhibits the enzyme, cyclooxygenase (COX). In one embodiment, the NSAIDs include selective COX-2 inhibitors such as celocoxib, refocoxib, and N-[2-(cyclohexyloxy)-4-nitrophenyl]methanesulfonamide.

Another group of useful therapeutically active agents are enzyme inhibitors. Examples of enzyme inhibitors include chrophonium chloride, N-methylphysostigmine, neostigmine bromide, physostigmine sulfate, tacrine HCL, tacrine, 1-hydroxymaleate, iodotubercidin, p-bromotetramisole, 10-(α-diethylaminopropionyl)-phenothiazine hydrochloride, calmidazolium chloride, hemicholinium-3,3,5-dinitrocatecho-1, diacylglycerol kinase inhibitor 1, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−)deprenyl HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-α-meth-ylbenzylamine (DCMB), 8,9-dichloro-2,3,4,5-tetrahydro-1H-2-benzazepine hydrochloride, p-aminoglutethimide, p-aminoglutethimide tartrate, R(+) p-aminoglutethimide tartrate, S(−).sub.3-iodotyrosine, alpha-methyltyrosine, L(−)alpha methyltyrosine, D,L(−)cetazolamide, dichlorophenamide, 6-hydroxy-2-benzothiazolesulfonamide, and allopurinol.

Another group of useful therapeutically active agents are anti-pyretics and antiinflammatory agents. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

Preferred electrospun fiber compositions of the invention include a growth factor, e.g., a nerve growth factor such as NGF or GNDF.

The compositions of the invention can be evaluated using a number of techniques. For example, the electrospun fiber compositions of the invention can be evaluated for the ability to release therapeutically active agents using in vivo or in vitro methods. For example, a composition of the invention may be allowed to incubate in a solution, e.g., an aqueous solution, for a prolonged period of time during which aliquots are removed and tested for the amount of therapeutically active agent released, and further, for the bioactivity of the agents. Alternatively, the compositions of the invention may be administered to a test animal, e.g., a rat, mouse, pig, or monkey, and levels of the therapeutically active agent can be monitored in, for example, the blood as a function of time.

Further, implantable films or tubes of the invention comprising electrospun fiber compositions comprising one or more therapeutically active agents can be surgically implanted into an animal model of the particular disease or conditions being tested. For example, the examples set forth a model for nerve regeneration that can be used to evaluate the efficacy of nerve guide conduits of the invention.

Methods of Making the Compositions of the Invention

The compositions of the invention can be made using methods that are known to one of ordinary skill in the art. The electrospun fiber compositions described herein can be made using electrospinning methods that are well known in the art and can be preformed using only routine experimentation.

Specifically, a charged solution comprising, for example, a polymer and one or more therapeutically active agents is fed through a small opening or nozzle (usually a needle or pipette tip). Due to its charge, the solution is drawn toward a grounded collecting plate, e.g., a metal screen, plate, or rotating mandrel, typically 5-30 cm away, as a jet. During the jet's travel, the solvent gradually evaporates, and a charged fiber is left to accumulate on the grounded target. The charge on the fibers eventually dissipates into the surrounding environment. If the target is allowed to move with respect to the nozzle position, specific fiber orientations (aligned or random) can be achieved.

The compositions of the invention can be made as electrospun fiber compositions, as electrospun fiber compositions on a substrate, e.g., a film, or as electrospun fiber compositions on the surface, e.g., the inner surface, of a tube.

Pharmaceutical Compositions

The invention also comprises pharmaceutical compositions comprising an electrospun fiber composition comprising a therapeutically effective amount of a therapeutic agent and, optionally, a pharmaceutically acceptable carrier. In particular embodiments, the compositions contain one or more biological therapeutics.

The pharmaceutical compositions of the invention provide the benefit of releasing biologically active therapeutic agents over an extended period of time.

In an exemplary embodiment, the pharmaceutical composition of the invention provides an electrospun fiber composition comprising an anti-inflammatory compound.

The pharmaceutical compositions of the invention may be formulated for administration in any convenient way for use in human or veterinary medicine. The pharmaceutical compositions of the invention include those suitable for topical, and/or parenteral administration. The pharmaceutical compositions of the invention may conveniently be presented in unit dosage form and may be prepared by any methods well known in the art of pharmacy. Administration can be systemic or local.

In one embodiment, the pharmaceutical composition is administered locally to the desired location. For example, in one embodiment an electrospun fiber composition comprising nerve growth factors is administered into the subarachnoid space after spinal cord injury. In another embodiment, the composition is introduced into the cerebrospinal fluid of the subject. In certain another embodiment, the composition is introduced intrathecally, e.g., into a cerebral ventricle, the lumbar area, or the cistema magna. In another embodiment the composition is introduced intraocullarly, to thereby contact retinal ganglion cells.

In another embodiment the composition is delivered locally to promote guided neurite elongation. Such methods are described herein, and include the use of nerve guide conduits, comprising electrospun fiber compositions comprising therapeutic agents, e.g., nerve growth agents.

The amount of biologically active ingredient(s) which can be incorporated into the electrospun fiber compositions of the invention to produce a single dosage form will vary depending upon the condition being treated, the host being treated, the particular mode of administration. The amount of active ingredient(s) which can be combined with the electrospun matrix material to produce a single dosage form will generally be that amount of the compound(s) which produces a therapeutic effect.

Methods of Treatment

The therapeutic electrospun fiber compositions of the invention can be administered to a subject by conventional routes of administration as described above. Alternatively, compositions of the invention that need surgical implantation can be implanted by surgical procedures known in the art. For example, nerve guide conduits of the invention can be implanted in a desired location using a suitable surgical procedure. Suitable surgical procedures are described, for example, in Hadlock et al., Archives of Otolaryngology—Head & Neck Surgery 124:1081-1086, 1998; WO 99/11181; U.S. Pat. No. 5,925,053; WO 88/06871; Wang et al., Microsurgery 14:608-618, 1993; and Mackinnon et al., Plast. Reconst. Surg. 85:419-424, 1990.

### EXAMPLES

It should be appreciated that the invention should not be construed to be limited to the examples that are now described; rather, the invention should be construed to include any and all applications provided herein and all equivalent variations within the skill of the ordinary artisan.

### Example 1: Sustained Release of Proteins from Electrospun Biodegradable Fibers

The following example provides exemplary methods for producing electrospun fiber compositions comprising biological therapeutics. The example further provides data demonstrating the sustained release of biologically active proteins from the electrospun fiber compositions.

### Materials

Recombinant human n-nerve growth factor (NGF) and DuoSet ELISA development system for human n-nerve growth factor were purchased from R&D Systems, Inc. A rat pheochromocytoma cell line, PC12, was obtained from American Type Culture Collection. Mouse collagen, Type IV, was purchased from BD Biosciences. Hepes buffer was obtained at a concentration of 1M from Cellgro. Phosphate buffered saline (PBS), pH 7.4, containing no calcium chloride and magnesium chloride; Fungizone Amphotericin B at a concentration of 250 μg/ml; penicillin-streptomysin (10000 U/ml); and RPMI medium 1640 with L-glutamine were obtained from GIBCO, Invitrogen Corporation. Dichloromethane (99.8% anhydrous) and albumin, fluorescein isothiocyanate conjugate bovine (FITC-BSA) were obtained from Sigma-Aldrich Corporation. The serum-free RPMI cell culture medium consists of RPMI 1640 medium, 1% Hepes buffer, 1% sodium pyruvate, 0.275% of penicillin-streptomysin, and 0.556% glucose. Sodium Azide, poly(ε-caprolactone) (PCL) and c-Caprolactone were purchased from Sigma-Aldrich Corporation. ε-Caprolactone was purified by vacuum distillation before use. Ethyl ethylene phosphate (EEP) was synthesized by a method described previously (Wen and Zhuo 1998).

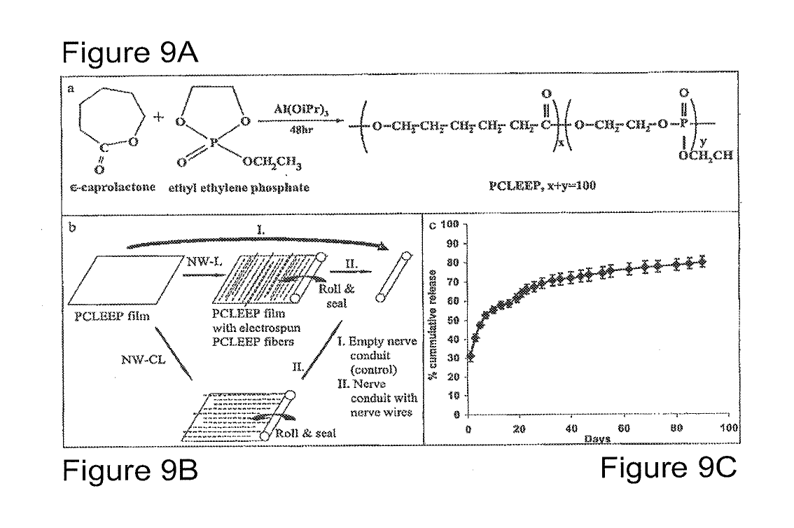

The PCLEEP copolymer with a 15 molar percent of EEP (M_{w}: 70,760, Mₙ: 25,800) was synthesized according to a procedure described by Wen et. al. (Wen and Zhuo 1998), as illustrated in Scheme 1 (FIG. 9A-FIG. 9C). Briefly, ε-Caprolactone and EEP were copolymerized in an ampoule using Al(OiPr)₃ as the initiator. After vacuum drying for 3 h, the ampoule was sealed and immersed in an oil bath at 100° C. for 48 h. The resulting polymer was dissolved in dichloromethane, washed with saturated NaCl solution three times, and then dried over Na₂SO₄. After quenching the solution into ether, the precipitated polymer was further purified by dissolving in acetone and quenching in distilled water.

Electrospinning of PCLEEP Fibers

The electrospinning parameters studied include: syringe tip-to-target distance of 5 cm to 10 cm, electrical voltage up to 20 kV, flow rate from up to 9.0 ml/min and polymer concentration from 2 to 12 wt % of PCLEEP in dichloromethane. For NGF-BSA encapsulated PCLEEP fibers, 100 μl of 100 μl g/ml of NGF reconstituted in 0.1 wt % of BSA was added into 30 μl of 10 wt % BSA. BSA, which was used as a filler protein, was dissolved in PBS. In the case of the FITC-BSA encapsulated PCLEEP fibers, in order to maintain similar mass and volume of BSA used in the NGF-BSA encapsulation, 123.75 μl of 2% FITC-BSA and 6.25 μl of 10% BSA solution was used. The resulting protein solution was added into 1.2 ml of 12 wt % of PCLEEP in dichloromethane polymer solution, giving a protein-polymer suspension, which was then vortexed to distribute the protein suspension uniformly throughout the polymer solution. The protein-polymer suspension was then dispensed at a flow rate of 8.5 ml/h at the beginning of the electrospinning process, in order to overcome the surface tension of the solution. The flow rate was subsequently decreased to 4.5 ml/h after electrospinning has started. The solution was dispensed using a syringe pump (KD Scientific), through a 30G syringe needle. The voltage applied to the syringe needle was 7.0 kV (Gamma High Voltage Research). The tip to target distance was 5-6 cm. The target comprised of an aluminum rotating drum 10 cm in diameter, rotating at 2200 rpm. Plain PCLEEP fibers were obtained by electrospinning 12 wt % of PCLEEP in dichloromethane polymer solution using similar electrospinning parameters, except with a smaller voltage of 5 kV.

Polymer and Fiber Characterization

Fiber Morphology

The electrospun fiber meshes with and without protein encapsulation were sputter coated with ˜2.5-3 nm of chromium (Denton vacuum, DV-502A) and were observed under the SEM (Leo field emission SEM, Leo 1530) at 1 kV.

Polymer Degradation

In vitro degradation study was conducted by placing fiber samples in 3 ml of distilled water with 0.01 wt % of sodium azide at 37° C. Three samples were used for each time point during this study, with each sample weighing about 32 mg and having dimensions 105 mm×12 mm×0.023 mm. The samples were withdrawn at predetermined time points, washed 3 times with distilled water and dried to constant weight under vacuum. The percentage weight change was determined as the ratio of change in mass to the original mass of the fiber sample. The change in molecular weight of the samples was determined using gel permeation chromatography (Shimadzu HPLC System, which comprises of Refractive Index Detector, RID-10A; System controller, SCL-10A VP; Liquid Chromatograph, LC-10AT).

Polymer Swelling Behavior

The swelling behavior of PCLEEP was evaluated by separately incubating four PCLEEP sheets, each weighing (99±3) mg, in 8.0 ml of distilled water with 0.01 wt % of sodium azide at 37° C. At predetermined time points, the sheets were gently tapped dry on paper and weighed. The swelling ratio was then taken as the ratio of mass increase to the original mass of the polymer sheet. Sheets of PCLEEP were used instead of fibrous mesh because of the large water uptake by the mesh, making it difficult to remove all excess water that may be trapped in between fibers from the fibrous mesh.

Protein Release Kinetics

Three samples that were electrospun using the same electrospinning parameters were used in this study. The three electrospun fibrous meshes, each weighing (84.2±10.5) mg, were each soaked in 12-well plates filled with 3.0 ml of serum-free RPMI. Fungizone was added at a dilution of 1:200 and the fibers were incubated under static conditions at 37° C. in the presence of 5% carbon dioxide. At various time points, 1.5 ml of supernatant was retrieved from the wells and an equal volume of fresh medium with fungizone was replaced. The concentration of NGF in the supernatant was then determined by the Duoset ELISA kit. At the end of 3 months, the fibers were dissolved in 1.0 ml of dichloromethane and any residual NGF was extracted into 1.0 ml of PBS for ELISA. Earlier experiments conducted to quantify the extraction efficiency of BSA from 3 wt % of PCLEEP dichloromethane solution showed that the extraction efficiency was constant at 27% regardless of the mass of BSA present. Therefore, assuming that the extraction efficiency of NGF is also independent of the mass of NGF present, the extraction efficiency was obtained as follows. Maintaining the NGF to BSA ratio used in electrospinning the NGF-encapsulated fibers, 5 μg of NGF was dissolved in 15 μl of 10 wt % BSA and added to 1.0 ml of PCLEEP dicholoromethane solution. The polymer solution contained 84 mg of PCLEEP, which is equal to the average mass of the electrospun fibers. NGF was then extracted using 1.0 ml of PBS and the extraction efficiency was then evaluated from the concentration of the extracted NGF as determined by ELISA. A portion of the collected supernatant was also used to test the bioactivity of the released NGF. The distribution of encapsulated FITC-BSA in PCLEEP fibers was observed using confocal microscopy (Ultraview™ LCI, Perkin Elmer).

Bioactivity of Released NGF

PC12 cells, which differentiate to a neuronal phenotype in the presence of bioactive NGF (Greene et al. 1976), were used to test for the bioactivity of the NGF released from the electrospun PCLEEP fibers. PC12 cells were cultured in collagen type IV-coated (at a concentration of 6-8 μg per cm²) 24-well plates at a density of 1×10⁴ cells/cm². A volume of 400 μl of the NGF supernatant from the PCLEEP fibers was added to each well of PC12 cells and serum-free RPMI was added to top up the medium volume to 1.0 ml per well. As a positive control, 8 μl of 50 μg/ml of NGF solution was added to the PC12 cell culture medium, and the total volume of medium was then topped up to 1.0 ml with serum-free RPMI. A negative control in which no NGF was added to the serum-free RPMI medium was also used. Each set of samples was repeated twice. Images of the PC12 cells were taken 3 days after the supernatant was added into the culture medium, and 5 non-overlapping areas were photographed per well. Percentage of PC12 cells differentiated into neurons was determined by counting number of cells forming neurites longer than one cell length. An average of 650 cells was counted in each well.

### Data Analysis

All data presented in this study are expressed as mean±standard error of mean. Statistical analyses were conducted on the fiber mass loss data and the fiber molecular weight changes using paired-sample t-test and Student's t-test respectively.

### Results & Discussion

Electrospinning of PCLEEP Fibers

The electrospinning parameters used to produce the NGF-encapsulation fibers were chosen from a set of optimized parameters obtained after carrying out a series of systematic studies on the effects of flow rate, polymer and protein concentration, voltage applied and tip-to-target distances on the spinnability, jet stability and morphology of the polymer fibers, with and without protein encapsulation. In order to obtain a more stable polymer jet, 12 wt % of polymer solution was used. However, using this polymer concentration, at an electric voltage up to 20 k V no fiber formation would result at flow rates below 8.5 ml/min at the start of the electrospinning process.

Due to the low amount of FITC conjugated to the BSA solution, there was no impact on the electrospinning process, allowing the same electrospinning parameters optimized for NGF-encapsulated fiber formation to be used for the encapsulation of FITC-BSA.

Polymer and Fiber Characterization

Fiber Morphology

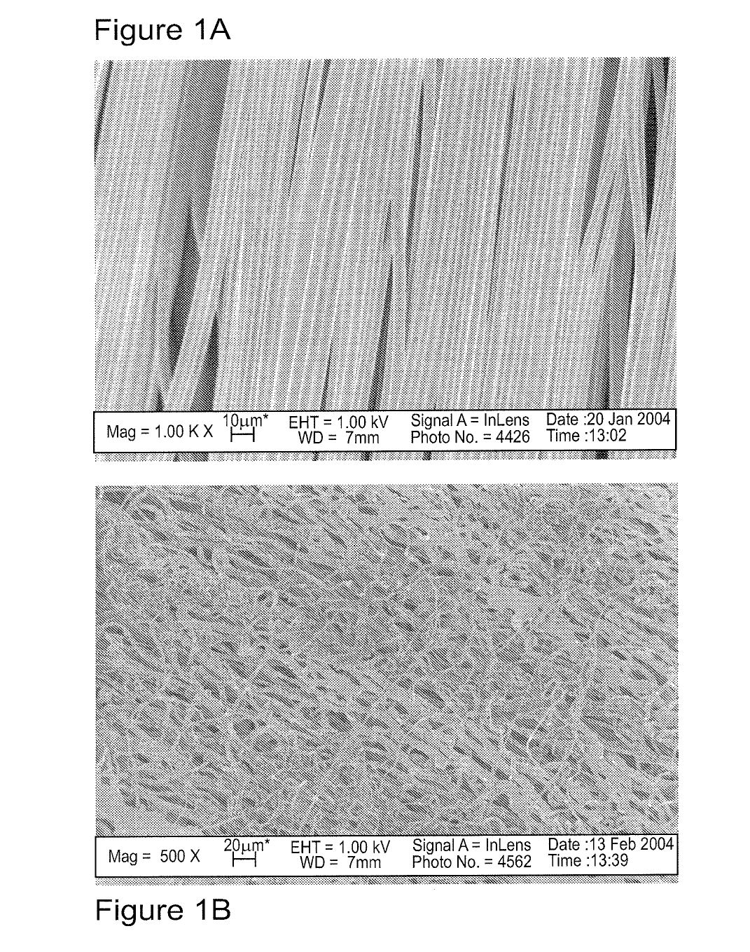

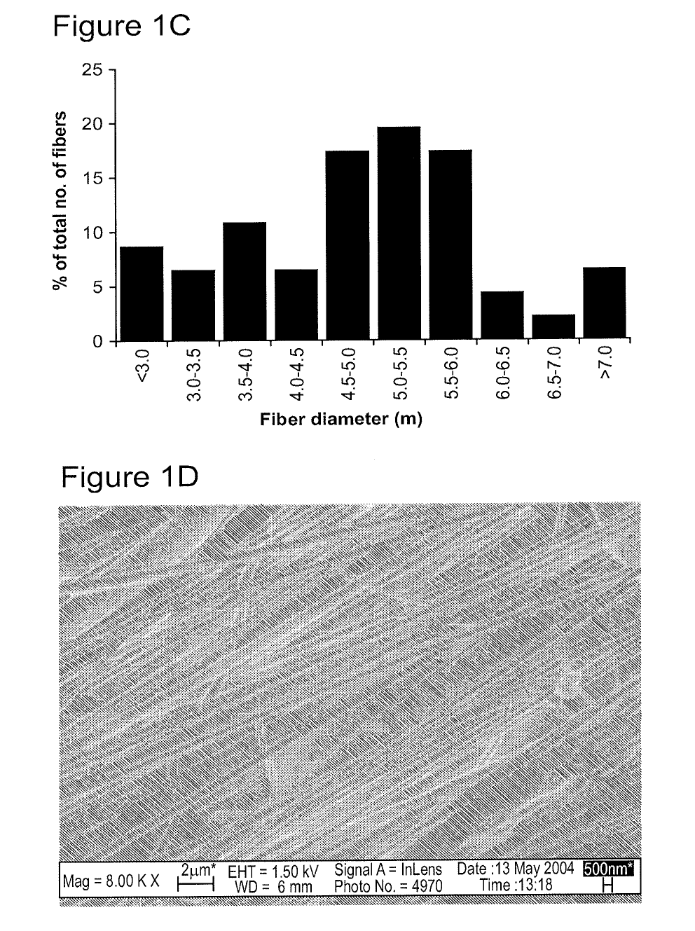

FIG. 1 shows the morphology of PCLEEP electrospun fibers with and without protein encapsulation. In the absence of protein solution, the polymer jet was steady; hence alignment of the fibers was easily obtained, as shown in FIG. 1a. In the presence of protein aqueous solution, however, due to the difference in the charge densities carried by the aqueous solution and the polymer solution, the electrostatic force acting on the solutions was different. The aqueous solution was observed to aggregate under the presence of the electric field, at the tip of the Taylor cone which was formed at the end of the syringe needle. The aggregation increased in size until it falls of the needle tip as a protein-encapsulated polymer aggregate, thus breaking the polymer jet during the electrospinning process. Fiber alignment relied on the matching of the rate of fiber deposition onto the rotating target, and the linear velocity of the target (Huang et al. 2003). Therefore, in the case of electrospinning plain PCLEEP, aligned fibers were obtained due to the matching of velocities. However, in the case of polymer-protein solution electrospinning, the jet was chaotic due to jet breakage, rendering it difficult to match the deposition rate and the linear velocity of the target. As a result, a more random fiber mesh was obtained.

A distribution of fiber diameter, such as that shown in FIG. 1c for plain PCLEEP fibers, was observed for all samples. The diameters lied in the micrometer range, with plain PCLEEP fibers having an average diameter of 5.01±0.24 μm; and protein encapsulated PCLEEP fibers an average diameter of 2.80±0.15 μm. The difference in diameters of the plain fibers and the protein-encapsulated fibers may be due to the difference in the stability of the polymer jet during electrospinning. The protein encapsulated fibers, being more unstable due to the difference in charge densities between the polymer and protein solutions, underwent more bending and whipping during electrospinning, hence possess a smaller fiber diameter.

This study aims to demonstrate the possibility of using electrospinning to fabricate protein-encapsulated fibrous scaffolds. Although the fibers obtained by the electrospinning process highlighted here are in the 1-10 micron range, it is possible to produce protein-encapsulated fibers in the submicron range, as shown in FIG. 1d. The fiber diameter is mainly determined by the flow rate and the polymer concentration (Zong et al. 2002: Fridrikh et al. 2003). Reducing the flow rate from 4.5 to 1 ml/h and the polymer concentration from 12 to 6% resulted in a reduction in fiber diameter. As shown in FIG. 1d, it was possible to obtain thinner fibers of an average diameter of 0.46±0.027 μm.

Polymer Degradation

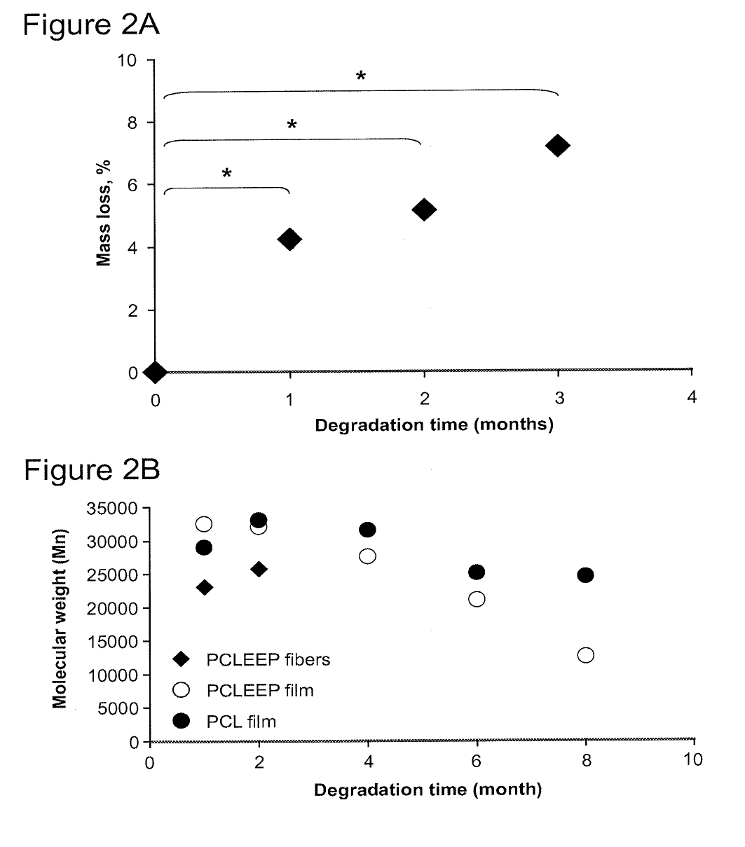

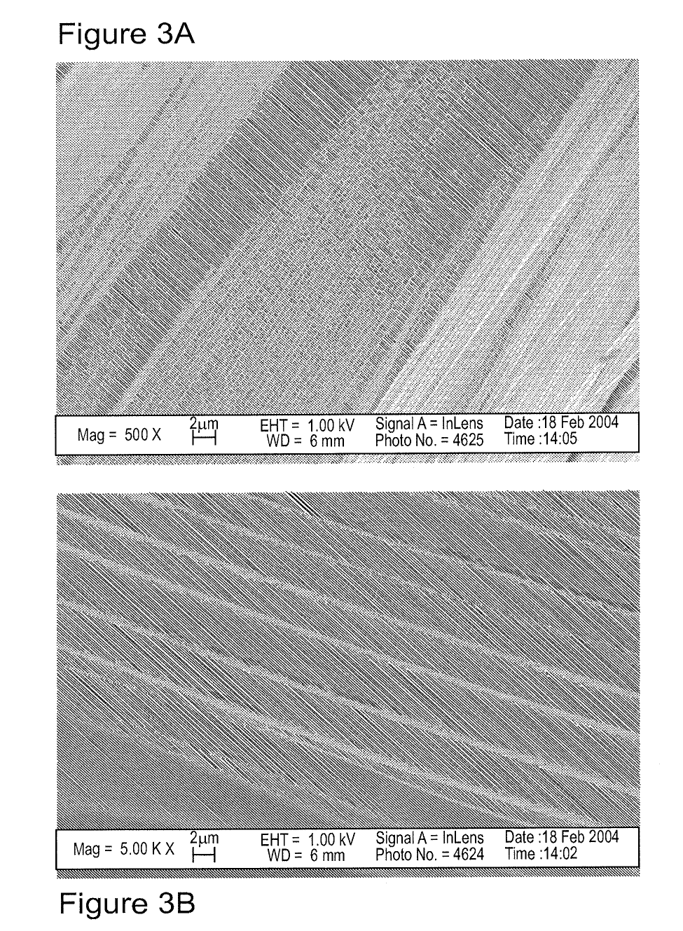

Although the aim of this study is to illustrate the feasibility of electrospinning in producing protein-encapsulated fibers, knowledge of the degradation of the polymer fibers helps in understanding of the protein release mechanism. The mass loss of PCLEEP during 3 months of incubation in distilled water at 37° C. is shown in FIG. 2a. Significant mass loss was observed after 1 month of incubation (p<0.01), although the changes in the Mₙ and M_{w} of the PCLEEP fibers were negligible after the first 2 months of incubation (p<0.01). The M_{w} of the PCLEEP fibers were 60, 360 and 64, 565 after 1 and 2 months of incubation respectively. The changes in the Mₙ of the PCLEEP fibers are shown in FIG. 2b. A comparison of the change in molecular weight of the PCLEEP fibers was also made with PCLEEP and PCL films. Both PCLEEP film and fibrous mesh showed no sign of decrease in molecular weight during the first 2 months of incubation. A higher degree of degradation compared to PCL could be seen for PCLEEP only after 6 months of incubation, presumably due to the presence of the phosphate bond in the backbone. With only a 15 molar % of EEP in the copolymer, however, the mass loss of the fiber sample was below 8% after three months of incubation. SEM inspection of the fibers after incubation in distilled water for 2 months showed no observable change in the fiber morphology, as shown in FIG. 3A and FIG. 3B.

Polymer Swelling Behavior

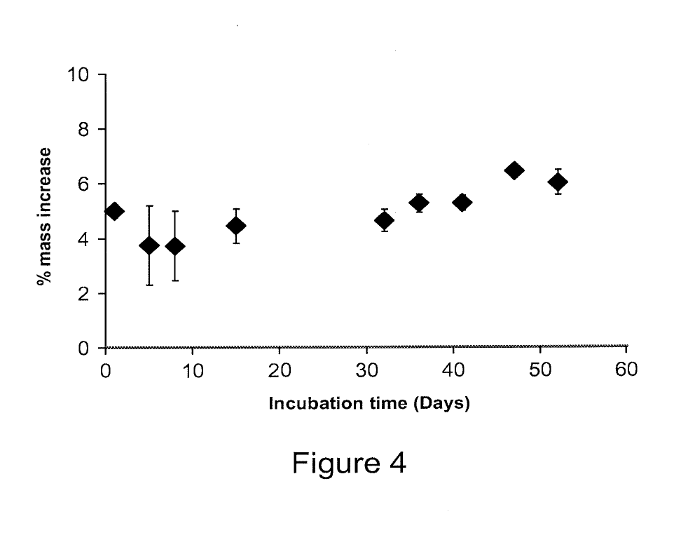

The swelling behavior of PCLEEP is shown in FIG. 4. After an initial 4.9±0.3% increase in mass within 24 hours the water content in the polymer maintained constant throughout the entire duration of the test.

Protein Release Study

Protein Distribution

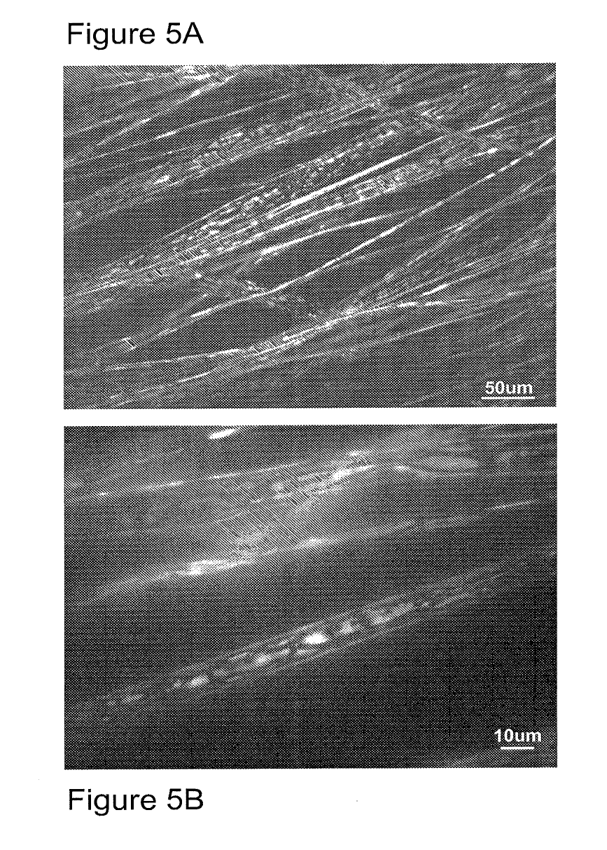

Protein distribution in the PCLEEP fibers was evaluated by observing the distribution of FITC-BSA encapsulated in the fibers, as shown in FIG. 5A and FIG. 5B. The volume and concentration of BSA used in fabricating the FITC-BSA encapsulated fibers was the same as that used in producing the NGF-BSA fibers for the protein release study. Hence, the distribution of FITC-BSA, in this case, is suggestive of the NGF-BSA distribution in the fiber. The protein was observed to be distributed in a uniformly random manner throughout the fibers in aggregate form. This may be due to phase separation between the organic polymer solution and the aqueous protein solution phases. Such an observation is similar to that found in the polymer-protein solution used during electrospinning, where the aqueous protein solution was distributed as suspension droplets throughout the polymer solution. The actual size and distribution of the suspension droplets may, however, differ from those of the protein aggregates found in the fibers due to possible coagulation of the aqueous phase during electrospinning.

Release Kinetics

The theoretical loading levels of NGF and BSA were 0.0123 and 4.08%, respectively. The actual loading level determined from the cumulative release profile of NGF was however only 3.10±0.53×10⁴%. The low loading efficiency is mainly caused by the instability of the polymer-protein jet during electrospinning. Due to the different charge densities in the aqueous protein solution and the polymer solution, the electrostatic forces acting on the solutions were different, thus causing the solutions to be dispensed at two different rates. The aqueous protein solution was observed to be dispensed at a faster rate. As a result, the protein solution was found to aggregate at the tip of the syringe needle during the electrospinning process, finally falling off the needle tip as protein-encapsulated polymer aggregates without being pulled into fibers. Therefore, the loading efficiency of the protein may be improved by using two separate flow rates for the polymer and protein solutions. Such may be achieved through the use of coaxial electrospinning (Li and Xia 2004; Sun et al. 2003; Huang et al. 2003).

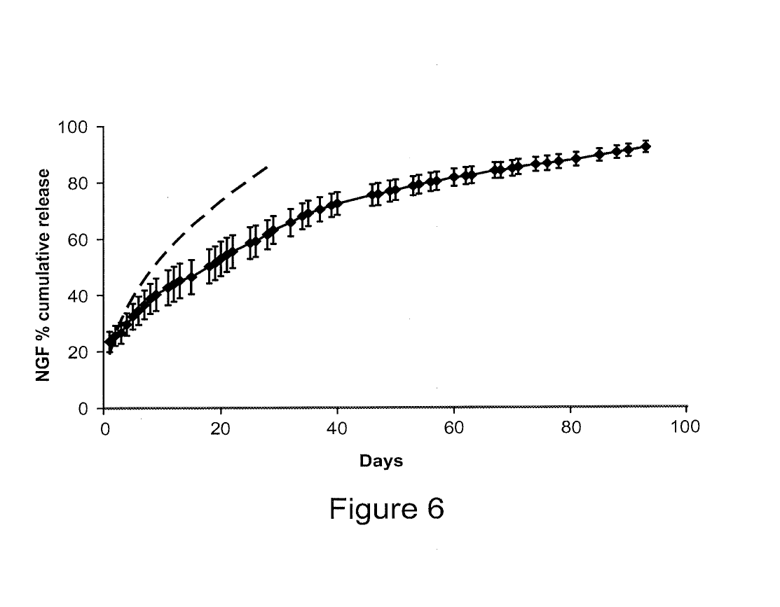

The NGF release profile is shown in FIG. 6. Sustained release of NGF from PCLEEP fibers was obtained for up to 3 months. After a modest burst of ˜20%, the protein was released in a relatively steady manner. The mechanism responsible for the relatively steady release of the protein after the burst is unclear. The observation that the fiber morphology and mass loss remained relatively unchanged in the first three months would suggest that diffusion is the predominant mechanism. In an attempt to analyze the diffusion mechanism in greater details, the aligned fibrous mesh system was modeled as a polydispersion of cylinders since a distribution of fiber diameter was observed as shown in FIG. 1c. The transport mechanism was compared with an ideal case of a monodispersion of cylinders. According to Ritger and Peppas (Ritger and Peppas 1987), assuming one-dimensional diffusion under perfect sink conditions, a generalized equation describing the transport of drugs from non-swellable devices may be expressed as:

$$\frac{{M}_{t}}{{M}_{∞}} = {kt}^{n}$$

Where Mₜ 15 me mass of drug released at time, t; M_{∞} is the mass of drug released as time approaches infinity; k is a constant and n is the diffusional exponent. Modeling the fibrous mesh as an array of parallel fibers, the length of each fiber is thus determined by the circumference of the rotating drum, which is approximately 31.4 cm. Furthermore, along with the fact that the average diameter of the electrospun protein encapsulated fibers is approximately 2.8 μm, the resulting high aspect ratio of the fibers allows one to assume one-dimensional diffusion of drugs from the electrospun fibrous mesh (Ritger and Peppas 1987). Observing the swelling behavior of PCLEEP in FIG. 4, after an initial 5.5±0.3% increase in mass within 24 hours, the polymer maintained its new dimensions throughout the entire duration of the test. This suggests that the rate of uptake of water by PCLEEP is negligible compared to the duration of the protein release test, which lasted for 90 days. Therefore, the

$$\frac{{M}_{t}}{{M}_{∞}} = 0.191 {t}^{0.34} {r}^{2} = 0.986$$

PCLEEP fibrous mesh was assumed to be non-swellable, thus giving,
According to Ritger and Peppas, for one-dimensional Fickian diffusion of drugs from a monodispersion of cylinders (Ritger and Peppas 1987)

$$\frac{{M}_{t}}{{M}_{∞}} = {kt}^{0.45} where k = 0.191$$

k is a constant that incorporates the characteristics of the polymer system and the drug (Ritger and Peppas 1987), and was obtained by the curve fitting highlighted above. Comparison of the first 60% of the release profile of β-NGF from electrospun fibrous mesh to that of a monodispersion of cylinders is shown by the dotted line in FIG. 6. The deviation of the diffusional exponent, n, differing from 0.45 could be due to the following reasons. The electrospun fibrous mesh comprises a distribution of fiber diameters, and the dissolution of the protein aggregates may constitute an additional rate barrier. While the release mechanism remains to be elucidated, this study did demonstrate that at least at low loading levels, proteins can be released in a sustained manner from such electrospun fibers. This would augur well for tissue engineering applications where potent growth factors are concerned.

Bioactivity of NGF

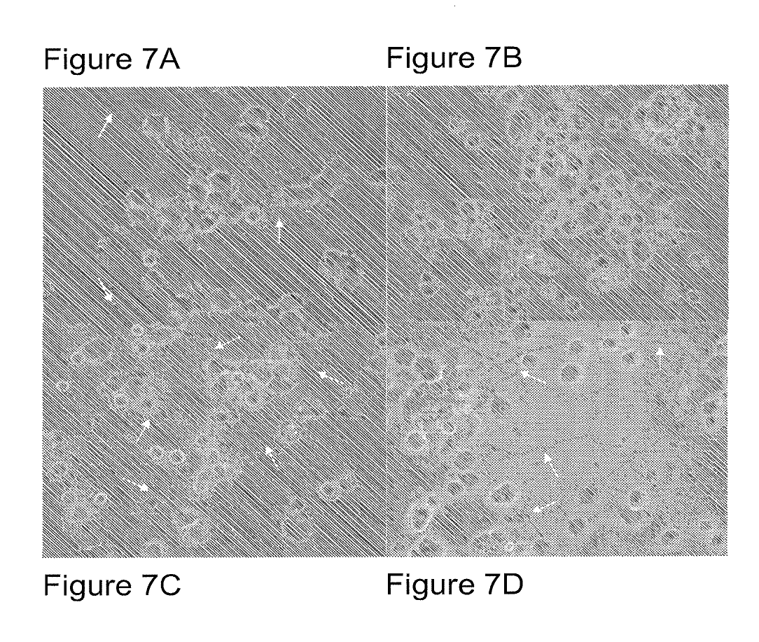

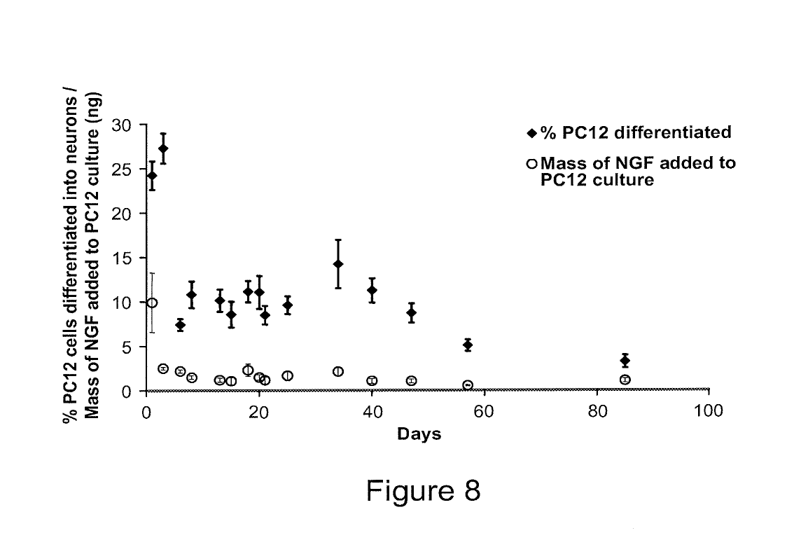

The bioactivity of the electrospun NGF was analyzed by observing the differentiation of PC12 cells into neurons, in the presence of the supernatant obtained from the electrospun NGF encapsulated fibers. The differentiation of the PC12 cells into neurons in the supernatant, and in the controls is shown in FIG. 7A, FIG. 7B, FIG. 7C, and FIG. 7D. The percentage of cells that showed signs of differentiation for various time points, together with the mass of released NGF added to each culture, is shown in FIG. 8. Since 1 ml of medium was used per well of PC12 cells, the concentration of released NGF in each well was equal to the mass of NGF added at each time point. The differentiation of PC12 cells is NGF dose-dependent (Green et al. 1978), although in a highly nonlinear manner with an S-shape relationship between the percentage of differentiated cells and the logarithmic function of the concentration of NGF (Katzir et al. 2002). The amount of released NGF added to the PC12 culture at time points beyond day 1 was in the range of 1-3 ng/ml, as determined by ELISA. Since the threshold for induction of PC12 cell differentiation is around 0.5 ng/ml (Thoenen and Bard 2002), this amount was enough to stimulate up to 15% of the PC12 cells. Although it is likely that the electrospinning process would have denatured the NGF, attempts to quantify the percent bioactivity retained by the NGF released from the fibrous mesh proved difficult. Culture-to-culture variability is typical in such cellular assays. Positive controls using fresh NGF at a concentration of 400 ng/ml would induce a wide margin of response of 20-55% of PC12 cells to differentiate, indicating the low sensitivity and non-linearity of this bioassay, similar to the observations made by others (Katzir et al. 2002; Pena et al. 1998). Nonetheless, this experiment did indicate that the NGF released from the PCLEEP fibers retained at least some degree of bioactivity for up to 3 months. Such a sustained release of NGF is useful for drug delivery applications, as NGF is known to have a short half-life in vivo, such as an elimination half-life of less than 5 hours in adult rats (Trai et al. 1994).

### Summary

Electrospinning has been successfully demonstrated as a practical way of fabricating biologically functional tissue scaffolds through the encapsulation of bioactive NGF. A sustained release of NGF from electrospun fibrous mesh for up to 3 months was obtained. The NGF released at the end of the 3-month period was still bioactive in stimulating PC12 cells into neurons.

### Example 2: Nerve Guide Conduit

Peripheral nerve regeneration and functional recovery is often disappointing over long lesion gaps despite surgical interventions and entubulation of the injured nerve. By far, the most common and efficient method of treatment is the use of autografts for long lesion gaps. However, drawbacks such as requirement of a second surgery, lack of available donor nerves, loss of donor nerve function, neuroma formation, and unacceptable scarring (Wang, Cai et al. 2002; Francel, Smith et al. 2003; Bunting, Silvio et al. 2005) justify the continuing search for better alternatives. The use of empty synthetic nerve guides has been one of the popular choices. These synthetic tubes, however, are only successful in bridging short nerve gaps such as ≤10 mm in the rat model (Ceballos, Navarro et al. 1999; Arai, Lundborg et al. 2000; Wang, Cai et al. 2002; Ngo, Waggoner et al. 2003; Cai, Peng et al. 2004). Additionally, there appears to be a species-dependent critical defect gap size, e.g. 15 mm in rats, beyond which the regeneration of injured nerves seldom occurs in these empty synthetic nerve guides (Ceballos, Navarro et al. 1999; Francel, Smith et al. 2003; Udina, Rodriguez et al. 2004).

In this experiment, we evaluate the approach of using electrospun fibers to provide both the contact guidance and growth factor signals. Nerve guide conduits composed of a biodegradable copolymer of caprolactone and ethyl ethylene phosphate (EEP), poly(ε-caprolactone-co-ethyl ethylene phosphate) (PCLEEP), with aligned GDNF-encapsulated electrospun PCLEEP fibers acting as nerve wires were fabricated. Electrospinning, a fiber spinning process that easily mass produces fibers with diameters ranging from nano- to micro-meter, has been widely used in the field of biomedical engineering over the past few years as wound dressings, tissue scaffolds and drug delivery vehicles for in vitro studies (Huang, Nagapudi et al. 2001; Stitzel, Pawlowski et al. 2001; Barras, Pasche et al. 2002; Kenawy, Bowlin et al. 2002; Matthews, Wnek et al. 2002; Luu, Kim et al. 2003; Matthews, Boland et al. 2003; Sanders, Kloefkorn et al. 2003; Wnek, Can et al. 2003; Yoshimoto, Shin et al. 2003; Boland, Matthews et al. 2004; Jin, Chen et al. 2004; Min, Lee et al. 2004). Encapsulation of drugs (Chew, Hufnagel et al.; Kenawy, Bowlin et al. 2002; Zeng, Xu et al. 2003; Jiang, Fang et al. 2004; Kim, Luu et al. 2004) and proteins (Chew, Hufnagel et al.; Chew, Wen et al. 2005) can also be achieved via electrospinning. Although a highly versatile and simple technique, the application of electrospinning for in vivo tissue engineering is still uncommon. The electrospun fibers used in the present study are at least an order of magnitude smaller than the aligned inclusions used in previous studies and have the added advantage of releasing neurotrophic factor in a sustained manner. The approach taken here effectively combines biochemical and topographical cues for enhanced sciatic nerve regeneration across a 15 mm critical defect in rats. The significance of the topographical cues provided by the aligned electrospun nerve wires on enhancing sciatic nerve regeneration is also addressed in this study.

### Materials and Methods

Materials

Recombinant human glial cell-derived neurotrophic factor (GDNF), 5 mg/ml, was provided by Amgen, Inc. Duoset ELISA development system for human glial cell-derived neurotrophic factor was purchased from R&D Systems, Inc. MicroBCA™ Protein Assay Reagent Kit was purchased from Pierce Biotechnology, Inc. Phosphate buffered saline (PBS), pH 7.4, containing no calcium chloride and magnesium chloride was purchased from GIBCO, Invitrogen Corporation. Sucrose, sodium phosphate monobasic monohydrate and sodium phosphate dibasic anhydrous were purchased from J. T. Baker. A 0.2M of Sorrensons phosphate buffer solution, pH 7.4-7.6, was then made from a mixture of 0.552 w/v % of sodium phosphate monobasic monohydrate and 2.27 w/v % of sodium phosphate dibasic anhydrous in distilled water, at a volume ratio of 5:4 respectively. Dichloromethane (99.8% anhydrous), bovine serum albumin (BSA), sodium azide, paraformaldehyde, glutaraldehyde, ε-caprolactone, acid hematoxylin solution, eosin B solution were purchased from Sigma-Aldrich Corporation. Horse serum, heat inactivated, was obtained from Invitrogen Corporation. Tissumend II synthetic absorbable tissue adhesive was purchased from Veterinary Products Laboratories. Isoflurane was obtained from Atlantic Biomedical. Nylon black monofilament (10-0) and silk filament (6-0) were purchased from Surgical Specialties Corporation and Ethicon Inc. respectively. Stainless steel wound clips were purchased from Autoclips. Optimal cutting temperature (OCT) compound was obtained from Tissue-Tek. Mouse anti-rat CD68 and Alexa Fluor® 488 goat anti-mouse antibodies were purchased from Serotec, Inc. and Molecular Probes™ Invitrogen Detection Technologies respectively.

The PCLEEP copolymer with a 15 molar percent of EEP (M_{w}: 70,760, Mₙ: 25,800) was synthesized according to a procedure described by Wen et. al. (Wen and Zhuo 1998). The synthesis is illustrated in FIG. 1a. Briefly, ε-Caprolactone and EEP were copolymerized in an ampoule using Al(OiPr)₃ as the initiator. After vacuum drying for 3 h, the ampoule was sealed and immersed in an oil bath at 100° C. for 48 h. The resulting polymer was dissolved in dichloromethane, washed with saturated NaCl solution three times, and then dried over Na₂SO₄. After quenching the solution into ether, the precipitated polymer was further purified by dissolving in acetone and quenching in distilled water.

Nerve Guide Conduit Fabrication

The fabrication process of the nerve guide conduits is highlighted in FIG. 9b. A PCLEEP film was fabricated by subjecting 0.5 g of PCLEEP polymer to a uniaxial compression load of 8×10³ kg for 2 minutes at 65° C. For the experimental control group, the PCLEEP film was rolled and sealed with 8 wt % of PCLEEP-dichloromethane solution into a cylinder to serve as an empty PCLEEP nerve conduit. Nerve conduits with PCLEEP fibers acting as nerve wires were fabricated by electrospinning aligned PCLEEP fibers directly onto the PCLEEP film. Based on previous experiments (Chew, Wen et al. 2005), 12 wt % of PCLEEP in dichloromethane was used as the polymer solution to be electrospun. The PCLEEP film was mounted on a grounded aluminum drum, 10 cm in diameter, which was rotating at 2200 rpm. The distance between the polymer solution and the PCLEEP film was set at 5-6 cm. The polymer solution was dispensed at a flow rate of 6 ml/h and an electrical voltage of 8 kV was applied to the polymer solution. GDNF-encapsulated PCLEEP fibers were fabricated by electrospinning a mixture of protein and polymer solution. The protein solution comprised of 45 μl of GDNF (5 mg/ml) and 5 μl of 30 wt % of BSA in PBS, resulting in a GDNF theoretical loading level of 0.13 wt % in the polymer solution. Prior to electrospinning, the protein-polymer solution was vortexed to uniformly distribute the protein suspension throughout the polymer solution. The resulting solution required a dispense rate of 8 ml/h and 7.5 kV for electrospinning, while all other processing parameters were kept the same as those used for electrospinning the plain PCLEEP fibers. For each experimental group of nerve conduits with electrospun fibers, one PCLEEP film and 1.0 ml of polymer solution were used for electrospinning. The final composite of film and fibers was then rolled and sealed with 8 wt % of PCLEEP-dichloromethane solution into cylinders. The nerve wires were aligned either longitudinally (NW-L) or circumferentially (NW-CL). The nerve conduits were sterilized by ultraviolet radiation for 30 minutes prior to surgical implantation.

Nerve Guide Conduit Characterization

In Vitro Protein Release Kinetics

For the in vitro protein release study, aligned protein-encapsulated fibers were obtained by electrospinning 1.0 ml of polymer-protein solution directly onto the grounded rotating aluminum drum without a polymer film. Similar processing parameters as those highlighted above, were used for electrospinning.

Each aligned protein-encapsulated fibrous mesh, weighing 50.7±4.9 mg, was incubated in 3.0 ml of PBS with 0.01 wt % of sodium azide at 37° C., under static conditions, in the presence of 5% carbon dioxide (n=4). At various time points, 1.5 ml of supernatant was retrieved from the wells and replaced with the same volume of fresh PBS with sodium azide. The concentration of GDNF was determined using the Duoset ELISA kit, following the manufacturer's protocol, with the exception that the standard curve was plotted based on various known concentrations of the GDNF obtained from Amgen Inc. The concentration of BSA was determined using the MicroBCA™ assay kit by assuming that the mass of GDNF released was negligible compared to that of the released BSA, which is 2 to 3 orders of magnitude larger than the mass of GDNF loaded.

At the end of 3 months, the fibers were dissolved in 1.0 ml of dichloromethane and any residual GDNF was extracted into 1.0 ml of PBS for ELISA. The GDNF extraction efficiency was obtained by extracting various known masses of GDNF loaded into a same concentration of PCLEEP solution (50.7 mg/ml). The mass of GDNF used ranged from 5 to 500 ng. Assuming that the mass of BSA has negligible effect on the extraction efficiency of GDNF, for each data point, the known mass of GDNF was mixed together with ˜1.6 mg of BSA (3.7 μl of 30 wt % of BSA), resulting in a constant volume of 4.741 of GDNF-BSA solution prior to adding into the polymer solution. The mass of BSA loaded was obtained by assuming 100% loading efficiency during electrospinning and estimating the mass of residual BSA left in the electrospun fibers after 3 months of incubation, based on the BSA release profile obtained. The protein-polymer solution was vortexed to ensure uniform distribution of the protein suspension. Thereafter, 1.0 ml of PBS was added to extract the protein. The total amount of residual GDNF in the electrospun fibers was then calculated after ELISA assay and accounting for the efficiency of the extraction method.

Structure & Appearance of Nerve Guide Conduits

Nerve guide conduits with and without nerve wires were sputter-coated with ˜2.5-3 nm in thickness of chromium (Denton Vacuum, DV-502A), prior to observation under the scanning electron microscope (Leo Field Emission SEM, Leo 1530). The accelerating voltage used was 1 kV. The average diameter of the electrospun fibers was determined by measuring at least 50 fibers using ImageJ 1.30 v (National Institutes of Health, USA)

In Vivo Experiments

Surgical Procedure

Thirty-four adult female Sprague-Dawley rats approximately 3.5 months of age were divided into 4 groups, receiving either empty PCLEEP nerve conduits (control, n=6); nerve conduits with plain electrospun PCLEEP fibers aligned longitudinally (NW-L (no GDNF)), n=9); nerve conduits with plain electrospun PCLEEP fibers aligned circumferentially (NW-CL (no GDNF), n=10) or nerve conduits with GDNF-encapsulated PCLEEP electrospun fibers aligned longitudinally (NW-L (GDNF), n=9). The rats were anesthetized under isoflurane delivered at a flow rate of 1 L/min. The left sciatic nerve was then exposed through a posterior thigh muscle-splitting incision and 6 mm of the sciatic nerve was resected to obtain a 15 mm nerve lesion gap. The PCLEEP nerve conduit was sutured to the proximal stump with one 10-0 nylon monofilament suture stitch and the distal stump with one 6-0 silk filament suture stitch. All nerve conduits, length 16 mm, were filled with 10 μl of PBS immediately prior to implantation. To ensure secured position of the nerve guide conduit, Tissumend II synthetic absorbable tissue adhesive was applied to the ends and the external surface of the center of the nerve conduit. One suture stitch of 10-0 nylon monofilament and stainless steel wound clips were then used to close the wound.

Electrophysiology—Motor Evoked Responses

At 1, 2 and 3 months post-operation, electrophysiological recovery was assessed using motor evoked responses. All animals were first anesthetized under isoflurane (flow rate 1 L/min) prior to the test. Compound motor action potential (CMAP) recordings in the tibial nerve innervated intrinsic foot muscles were recorded after stimulation of the sciatic nerve at the sciatic notch by needle electrodes as described before (Heine, Conant et al. 2004). Both CMAP readings from the left and right sciatic nerves were recorded for each rat.

Morphological Evaluation

Upon retrieval of the nerve conduits 3 months post-implantation, nerve cross-sections at 8-10 mm from the proximal end were processed for toluidine blue staining and transmission electron microscopy (TEM). The samples were fixed in a solution of 4 wt % paraformaldehyde and 3 wt % glutaraldehyde in PBS for 2 days before being transferred into Sorrensons phosphate buffer (0.2M). The tissue sections were further post-fixed with 2% of osmium tetroxide for 2 hours and dehydrated through 50%, 70%, 80% 95% and 100% ethanol solutions prior to mounting in embedding resin. Samples were then sectioned on an Ultracut E microtome at 1 μm thickness and stained with 1% toluidine blue for light microscopy. TEM samples were cut on a Reichert Ultracut S microtome in 0.5-0.65 μm thickness, placed on 0.5% formvar coated meshes and stained with 5% uranyl acetate and 0.3% lead citrate.

All samples that were stained with toluidine blue were imaged on a Nikon Eclipse TE2000-U microscope. Morphometric analysis of the nerve regeneration was then carried out by image analysis using ImageJ 1.30 v (National Institutes of Health, USA). Quantification of the total number of myelinated axons per cross section of each regenerated sciatic nerve was carried out by photographing the entire cross section of each nerve at 400× magnification with consecutive non-overlapping shots. The total number of myelinated axons and the total nerve area in each photograph was computed for the number of myelinated axons per nerve area (number density). The final number density for each sciatic nerve cross-section was then calculated as the average number density of all the photos taken for each sciatic nerve. The total number of myelinated axons per nerve cross-section was calculated from the product of the total cross-sectional area of the regenerated nerve and the average number density. Evaluation of the G-ratio, which is the ratio of axon diameter to the total diameter of the nerve fiber, was carried out by photographing randomly selected fields of each sciatic nerve cross-section at 1000× magnification. For each sample, at least 80 myelinated axons were measured.

For TEM observation of the sciatic nerve, cross-sections were viewed under the Hitachi H600 electron microscope, using an accelerating voltage of 75 kV.

Histological Evaluation

Three months post-implantation, nerve cross-sections at 5-8 mm from the proximal end were retrieved and immediately immersed into 4% paraformaldehyde for immunofluorescent and haematoxylin & eosin (H&E) staining. The samples were transferred into 15% and then 30% sucrose solutions after 24 and 48 h respectively. All samples were stored at 4° C.

For macrophage staining, the sciatic nerve samples were mounted in OCT and sectioned at 20 μm thickness for immunostaining. Following cryostat sectioning, the samples were post-fixed in 4% paraformaldehyde in PBS for 30 minutes. The samples were then transferred into 0.2% Triton X in PBS for 30 minutes and then blocked in 10% horse serum for 2 h. Mouse anti-rat CD68 (ED1) antibody was then diluted 1:1000 using 1% horse serum. The samples were incubated in the primary antibody at 4° C. overnight to stain for activated macrophages. Thereafter, samples were transferred into goat anti-mouse (AlexaFluor 488) secondary antibody diluted 1:1000 in 1% horse serum and DAPI (1:2000 dilution) for 1 h of incubation. All incubation steps, except overnight incubation, were carried out at room temperature. The samples were rinsed three times in PBS in between each step. All samples were finally imaged on a Perkin Elmer UltraVIEW spinning disk confocal microscope.

For H&E staining, the sciatic nerve samples were mounted in OCT and cryostat-sectioned at 10 μm thickness followed by standard H&E staining. All samples were viewed under the Olympus BX51TF upright microscope.

Data Analysis

All data presented in this study are expressed as mean±standard error of mean (SEM). One-way ANOVA followed by the Fisher's Least Significant Difference (LSD) method were used for the statistical analysis of nerve cross-sectional area. Fisher-Irwin test was used for the analysis of the percentage of rats with electrophysiological recovery. All other statistical analyses were carried out using the Kruskal-Wallis test followed by the Mann-Whitney U test.

### Results

In Vitro Protein Release Kinetics

FIG. 9c shows the in vitro release profile of encapsulated GDNF from PCLEEP electrospun fibers. After an initial burst release of about 30% of GDNF, the remaining protein was released in a fairly sustained manner for almost 2 months before leveling off.

Structure & Appearance of Nerve Guide Conduits

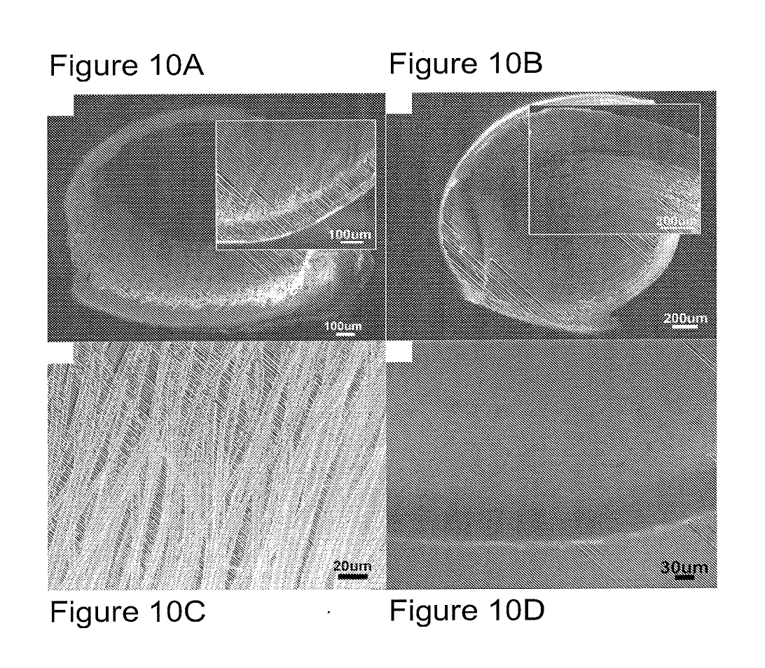

FIGS. 10a and 10b shows the cross sections and the inner surfaces of nerve conduits with longitudinally and circumferentially aligned electrospun fibers respectively. The average inner diameter and wall thickness of the nerve guides are 1.5±0.2 mm and 83.2±2.9 μm, respectively. The different fiber arrangement in the two samples is more clearly illustrated by the insets of FIGS. 10a and 10b. FIG. 10c shows the alignment of the electrospun fibers on the inner surface of nerve guides with nerve wires. The diameters of plain and GDNF-encapsulated PCLEEP fibers are 5.08±0.05 μm and 3.96±0.14 μm respectively. The inner surface of the control group is generally smooth as shown in FIG. 10d.

Morphological Evaluation

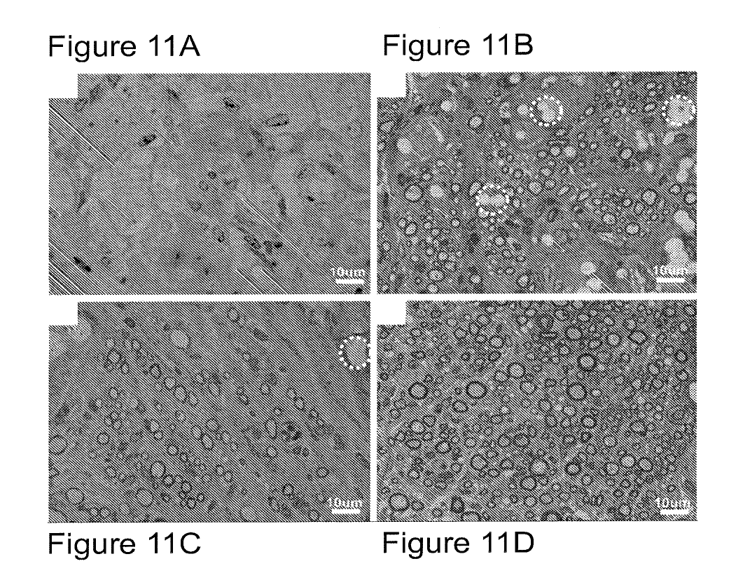

Light micrographs of the cross-sections, 8-10 mm away from the proximal end, of the regenerated sciatic nerve are shown in FIG. 11A, FIG. 11B, FIG. 11C, and FIG. 11D. Bridging of the 15 mm defect by the regenerated sciatic nerve was observed in all the rats that received nerve guides with nerve wires, as opposed to only 3 out of 6 in the group with empty nerve guides. Only 4 out of 6 rats in this control group had regenerated sciatic nerve at 8-10 mm from the proximal end, out of which, only 2 rats had myelinated axons in the regenerated sciatic nerve at the same location.

Empty spaces taking the shape of fiber bundles, such as those identified by the dashed circles in FIG. 11b, were observed in the cross-sections of the regenerated sciatic nerves from the NW-L (no GDNF) group. The empty spaces are likely to be of those occupied by bundles of electrospun fibers that remained at the site of injury, but dissolved during histology sample processing. Similar voids were also observed in the cross-sections of the sciatic nerves from the NW-CL (no GDNF) group, as indicated by the dashed circle in FIG. 11c. Protein encapsulated nerve wires, however, were not found in any of the cross-sections of the sciatic nerves from the NW-L (GDNF) group.

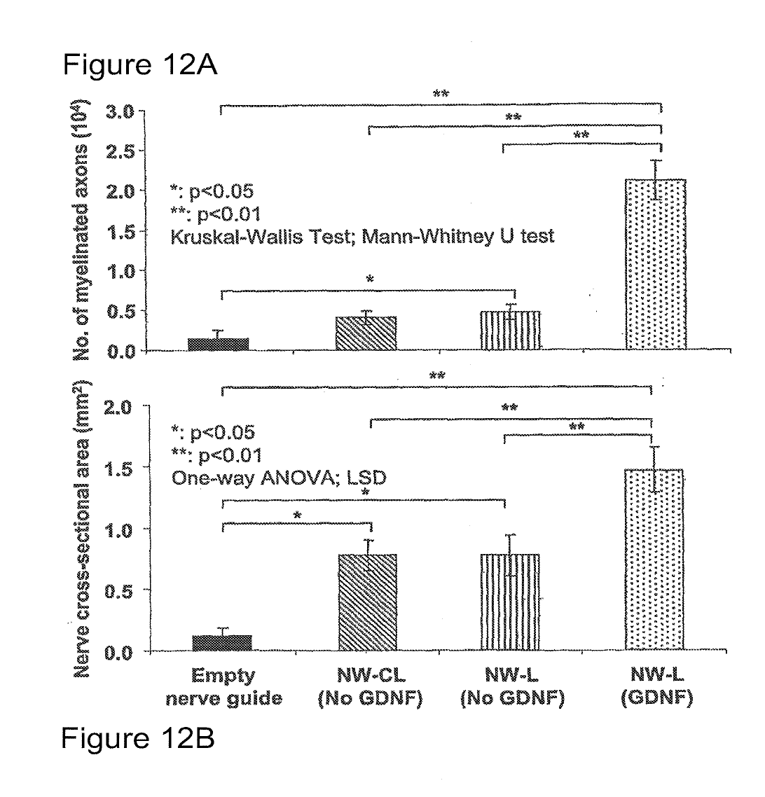

The total number of myelinated axons and the cross-sectional area of the regenerated sciatic nerve at 8-10 mm from the proximal end of the nerve conduit for each experimental group are shown in FIG. 12a and FIG. 12b, respectively. With the inclusion of electrospun fibers, either longitudinally or circumferentially aligned but without GDNF, the number of myelinated axons and the cross-sectional area of the regenerated nerves significantly increased as compared to the empty conduits. There is however no significant difference between the different orientation of the aligned fibers. The introduction of exogenous growth factor further improved nerve regeneration significantly.

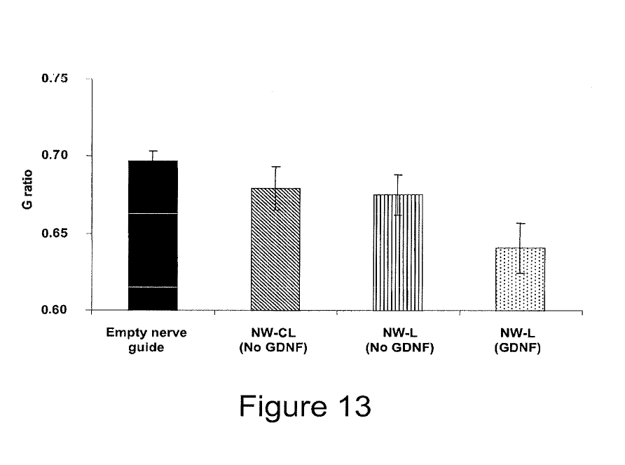

FIG. 13 shows the G-ratio of the experimental groups. In the case of the group with empty conduits, the G ratio was computed based on the 2 animals that had myelinated axons at 8-10 mm from the proximal end of the nerve guide. No significant difference in G-ratio was observed between the experimental groups.

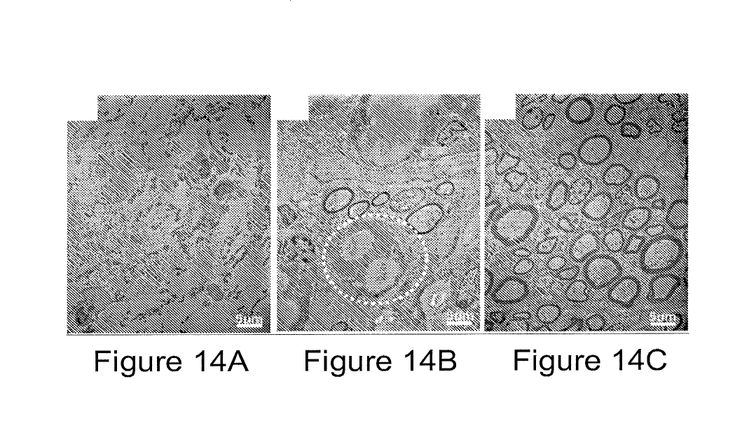

The typical TEM micrographs of the cross-sections of the regenerated sciatic nerves are shown in FIG. 14A, FIG. 14B, and FIG. 14C. The regenerated nerves in the control group consisted mainly of fibrous tissues, with little or no myelinated axons observed, as shown in FIG. 14a. On the contrary, large numbers of myelinated axons were found in other experimental groups. FIG. 14b illustrates the tendency of myelinated axons regenerating in close proximity to the PCLEEP nerve wires, which are identified and enclosed in the dashed circle.

Immunofluorescent Staining and Histological Evaluation

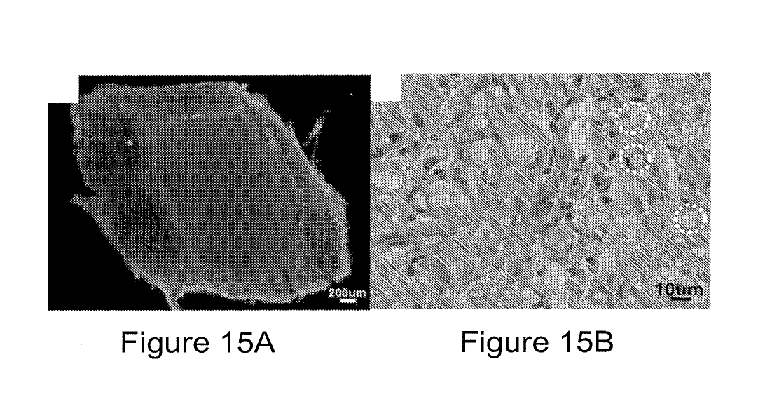

FIG. 15a shows the fluorescent micrograph for ED1 immunostaining of the cross-section of a regenerated sciatic nerve, 5-8 mm from the proximal end. For all experimental groups, activated macrophages were found mostly along the periphery of the regenerated sciatic nerve, where it is in close contact with the nerve conduit. The H&E staining of the cross-sections of the regenerated sciatic nerves from experimental groups that received nerve guides with plain nerve wires is shown in FIG. 15b. The micrograph revealed the absence of an acute immune response in close proximity to the nerve wires (identified by dashed circles), indicating the non-inflammatory nature of the nerve wires.

Electrophysiological Assay—Evoked Motor Responses

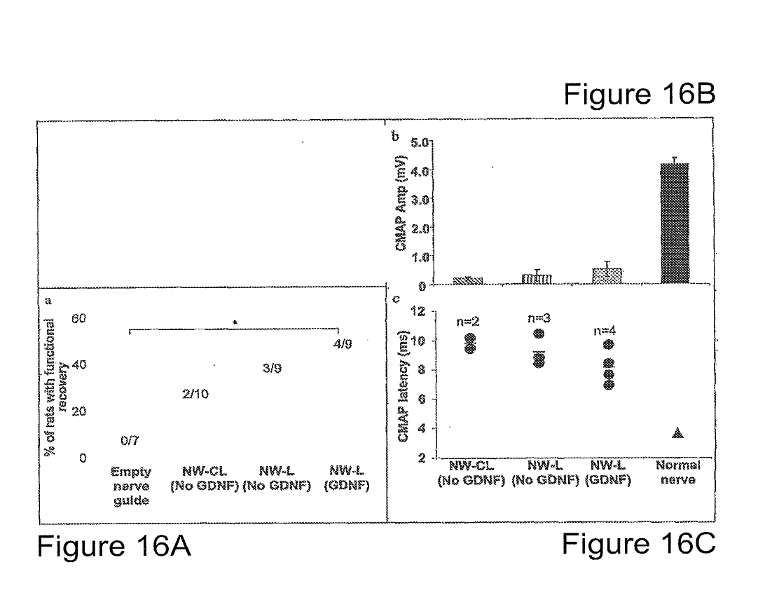

Evoked motor responses at 1 and 2 months post implantation revealed no recovery in any of the rats. However, electrophysiological recovery was observed 3 months post-implantation. The inclusion of GDNF-encapsulated nerve wires led to partial functional recovery in four out of nine rats (FIG. 16a). Although not statistically significant, the inclusion of plain nerve wires also resulted in functional recovery in a portion of rats as compared to none in the group without wires. Two out of ten rats and three out of nine rats in the NW-CL (no GDNF) and NW-L (no GDNF) groups respectively showed electrophysiological recovery.

FIGS. 16b and 16c illustrates the amplitudes of the CMAP and their corresponding latency respectively. The amplitude and the latency of the CMAP of the animals that received GDNF-encapsulated nerve guides appeared to be better than those receiving nerve guides with plain nerve wires, although the results are not statistically significant due to the small number of animals that showed functional recovery in each group. The values of the amplitude and latency remain inferior to a normal nerve. However, these values are expected to approach that of a normal nerve with respect to time.

### Discussion

Poly(ε-caprolactone) is a biodegradable and biocompatible polymer that has been widely studied for medical device and drug delivery applications (Huatan, Collett et al. 1995; Medlicott, Tucker et al. 1996). Its low degradation rate, however, makes it less optimal for some tissue scaffolding applications. The addition of a phosphate group to the polymer backbone was previously shown to enhance the biodegradability and flexibility of the polymer, thereby making it more suitable for nerve regeneration applications. The use of a biodegradable material is favorable because of the elimination of the need for a second surgery and the possible enhancement in nerve regeneration as compared to permanent nerve conduits due to the improved transportation of nutrients and the increased flexibility of the nerve conduit as the material degrades (Wang, Cai et al. 2002). The possibility of encapsulating and releasing at least partially bioactive proteins from electrospun PCLEEP (Chew, Wen et al. 2005) further reinforces the choice of this polymer as a material for the nerve guide conduits used in this study. Since a sustained release of partially bioactive NGF could be obtained from this biodegradable copolymer for a period of 3 months in vitro (Chew, Wen et al. 2005), the same polymer and technique of fabricating protein-encapsulated fibers were used in this Example for the fabrication of the nerve guide conduits.

Growth factors typically are labile. For example, the biologic half-lives of platelet-derived growth factor (PDGF), basic fibroblast growth factor (bFGF) and vascular endothelial growth factor (VEGF) are 2, 3 and 50 minutes respectively when intravenously administered (Chen and Mooney 2003). As a result, the use of polymeric drug delivery vehicles in the form of microsphere, nanosphere, and hydrogel to maintain a sustained localized delivery to the target site is attractive (Tornqvist, Bjorklund et al. 2000; Barras, Pasche et al. 2002; Bensadoun, Almeida et al. 2003). The release kinetics of the growth factors may also be controlled through the proper design of the delivery vehicle, catering to the specific needs of the target tissue injury or disease (Chen and Mooney 2003). Although Schwann cells can be used as a source of growth factor, the isolation and expansion requirement may be a potential drawback of this approach for practical usage (Rangappa, Romero et al. 2000; Rosner, Siegel et al. 2003; Mimura, Dezawa et al. 2004). In this Example, polymeric electrospun fibers were chosen as the delivery vehicle for the sustained release of GDNF.

Based on previous experiments (Chew, Wen et al. 2005), the presence of a large volume ratio of protein solution in the polymer solution to be electrospun can lead to frequent jet breakages during electrospinning. This in turn leads to a low loading efficiency of protein into the electrospun fibers. Therefore, the total volume of protein solution used in this study was restricted to 50 μl in order to minimize jet breakages during electrospinning. Being highly potent by nature, only minute amounts of growth factors (picograms to nanograms) is often required to elicit biological activity. The efficiency of growth factors in eliciting biological responses has also been found to be concentration and time-dependent (Chen and Mooney 2003). While the exact concentration of GDNF required to elicit a biological response in the rat model using our experimental approach is unknown, the amount of GDNF loaded was the maximum amount that could be used given the experimental restrictions imposed by the concentration of the available GDNF stock solution and the electrospinning process.

As demonstrated in Example 1, a sustained release of bioactive proteins from electrospun PCLEEP fibers was obtained for at least 3 months. In order to obtain similar results, the electrospinning parameters used in this study were maintained as close as possible to those used previously. BSA was used as a filler protein. In attempt to maximize the amount of GDNF that can be loaded into the polymer solution while maintaining a similar loading level of BSA as in previous experiments (Chew, Wen et al. 2005), 5 μl of 30 wt % of BSA was used.

Morphological Analysis

With the inclusion of aligned electrospun fibers, the number of myelinated axons and the cross-sectional area of the regenerated nerves significantly increased as compared to the control group of empty nerve conduits. This may be due to the contact guidance provided by the longitudinally aligned fibers, along with the increase in surface area available for cell attachment and growth. The control group of circumferentially aligned fibers, NW-CL (no GDNF), was an attempt to uncouple these two factors. As morphometric analysis revealed no significant differences, the conclusion leans towards a more adhesive surface rather than the effects of contact guidance.

The further significant improvement in nerve regeneration with the addition of exogenous GDNF demonstrates the effectiveness of the growth factor in enhancing sciatic nerve regeneration. The improved nerve regeneration in the presence of GDNF may also be attributed to the possible increase in macrophage invasion in response to the presence of the human protein during early stages of recovery. The macrophage invasion is manifested in the faster degradation of the nerve wires. Since macrophages have been found to release cytokines such as interleukin-1 (IL-1) that stimulates NGF production from cells like Schwann cells (Ngo, Waggoner et al. 2003), this may in turn add to the GDNF effect.

A normal rat sciatic nerve contains 7115±413 myelinated nerve fibers (Belkas, Munro et al. 2005). Clearly, the regenerated sciatic nerve in the empty conduit group is far inferior to a normal nerve even after 3 months of recovery. The number of axons in the groups that received plain nerve wires, aligned in either direction, is close to that of a normal sciatic nerve. In contrast, the total number of axons in the NW-L (GDNF) group lies well above the normal. The larger than normal number of myelinated axons in a regenerated nerve is not uncommon. It has been observed that the number of regenerated nerve fibers can be larger than the normal number even after 7 months of recovery (Ceballos, Valero-cabre et al. 2002; Francel, Smith et al. 2003). This is because, after injury, regenerating neurons can support multiple branching from the site of injury so as to maximize the possibility of each neuronal cell to reach its target organ. The excess sprouts will then be eliminated through axonal pruning due to the lack of survival signal from the target organ (Terenghi 1999) and will then help improve the correct reconnection of the nerve and its appropriate target (Ceballos, Valero-cabre et al. 2002).

A G ratio of about 0.7 is ideal for nerve conduction (Stang, Fansa et al. 2005), and it hovers around 0.6-0.7 in normal uninjured nerves (Fansa, Dodic et al. 2003). From FIG. 13, the G ratios of the groups that received nerve wires lie well within the range of the normal nerve. In general, a decreasing trend in the G ratio was observed with the inclusion of nerve wires and the introduction of exogenous growth factor respectively, indicating an enhancement in maturation of the myelinated axons as compared to an empty nerve conduit.

Both light and electron microscopy revealed the presence of PCLEEP fibers in the nerve guides with plain nerve wires at the time of sacrifice. However, no GDNF-encapsulated fibers could be found in the NW-L (GDNF) group. While the exact reason behind this observation is unclear, it appears that a more pronounced immune response towards the encapsulated and released human GDNF from the fibers, as manifested by activated macrophages found in the periphery of the regenerated nerve, has accelerated the degradation of the fibers, leading to complete degradation of the protein-encapsulated fibers within 3 months.

Electrophysiological Assay—Evoked Motor Responses

The success in achieving electrophysiological recovery in a significant portion of animals highlights the contrast of this study from many others, where the inclusion of microfilaments of diameters much larger than the ones used in this study (diameter 20-100 μm) were used. In most other studies, the state of regeneration of the nerve was solely evaluated through morphometric analyses, which may not be sufficient in evaluating the potential of a nerve guide conduit in enhancing nerve regeneration. This is because functional recovery is not always guaranteed even though nerve regeneration has occurred, due to the failure of regenerating axons to reach the appropriate target (Rangappa, Romero et al. 2000). To the knowledge of the authors, most studies carried out to evaluate the potential of contact guidance in enhancing nerve regeneration (Ceballos, Navarro et al. 1999; Ngo, Waggoner et al. 2003; Cai, Peng et al. 2004; Yoshii, Shima et al. 2004; Bunting, Silvio et al. 2005) have not evaluated the functional recovery of the animals, even though in some cases, the period of recovery is longer than that covered in this study (Ngo, Waggoner et al. 2003). Although Dahlin and Lundborg (Dahlin and Lundborg 1999) demonstrated functional recovery in their animals via pinch test, the sciatic nerve defect size used in their study was 10 mm instead of the critical defect size used in this study. Arai, et. al. (Arai, Lundborg et al. 2000), on the other hand, have demonstrated functional recovery in rats over a 15 mm defect. In their study, functional assay was conducted by measuring the anterior tibial and gastorcnemius muscle forces generated by electrical stimulation of the sciatic nerve. In this study, however, the muscle action potential was measured at the most distal foot muscles after sciatic nerve stimulation, making our functional assay more stringent. Moreover, the commercially available polyamide, catgut, polydioxanone and polyglactin microfilaments used in Arai's study preclude the incorporation of protein delivery functions. In contrast, the fabrication highlighted in this study not only enables one to easily include growth factors into the electrospun fibers, but also produces fibers of dimensions at least one to two orders of magnitude smaller. Besides proteins, drugs (Chew, Hufnagel et al.; Kenawy, Bowlin et al. 2002; Zeng, Xu et al. 2003; Jiang, Fang et al. 2004; Kim, Luu et al. 2004) and even DNA (Luu, Kim et al. 2003) may also be easily incorporated into the nerve wires via electrospinning.

Contact Guidance vs. Surface Area Effect

As with most other studies, this study took off with the aim to introduce contact guidance to enhance nerve regeneration. The inclusion of nerve wires, however, not only introduces contact guidance but also inevitably increases the total surface area available for cell attachment and growth. Therefore, an attempt to separate the effects of these two factors was made by introducing circumferentially aligned electrospun fibers into the nerve guide conduits. The insignificant differences in the degree of nerve regeneration as indicated by morphometric and functional analyses between the orientation of the aligned fibers seems to suggest that contact guidance may not play as prominent a role as hypothesized. The possibility of contact guidance playing a significant role during the initial or early phase of nerve regeneration, however, cannot be eliminated. Ceballos et. al. (Ceballos, Navarro et al. 1999) reported enhanced sciatic nerve regeneration in mice using aligned collagen gel, as compared to random collagen gels, after 60 days post implantation. The alignment and elongation of cells on micro-(Thompson and Beuttner 2004; Schmalenberg and Uhrich 2005) and nano-topographies (Johansson, Carlberg et al. 2005) are also often observed in in vitro cell cultures within a period of 1 week.

### CONCLUSIONS

Significant enhancement in nerve regeneration through the use of electrospun fibers and the sustained release of exogenous growth factor, GDNF, was demonstrated in this Example by morphometric and functional analyses. The increase in surface area provided by the electrospun fibers for cell attachment and growth is the dominant factor in nerve regeneration as compared to contact guidance at 3 months post operation. On its own, increase in surface area can help in tissue regeneration; however, the synergistic effect of the encapsulated protein ensured a more significant recovery. This study also served to demonstrate the use of electrospinning as a simple and feasible method to easily include biochemical and topographical cues into a single implant to enhance peripheral nerve regeneration.

### REFERENCES

Arai, T., G. Lundborg, et al. (2000). “Bioartificial nerve graft for bridging extended nerve defects in rat sciatic nerve based on resorbable guiding filaments.” Scand J Plast Reconstr Hand Surg 34: 101-108.
Aszmann, O. C., K. J. Korak, et al. (2002). “Simultaneous GDNF and BDNF application leads to increased motoneuron survival and improved functional outcome in an experimental model for obsteric brachial plexus lesions.” Plast. Reconstr. Surg. 110: 1066.
Barras, F. M., P. Pasche, et al. (2002). “Glial cell line-derived neurotrophic factor released by synthetic guidance channels promotes facial nerve regeneration in the rat.” Journal of Neuroscience Research 70: 746-755.
Belkas, J. S., C. A. Munro, et al. (2005). “Peripheral nerve regeneration through a synthetic hydrogel nerve tube.” Restorative Neurology and Neuroscience 23: 19-29.
Bensadoun, J.-C., L. P. d. Almeida, et al. (2003). “Comparative study of GDNF delivery systems for the CNS: polymer rods, encapsulated cells and lentiviral vectors.” Journal of Controlled Release 87: 107-115.
Boland, E. D., J. A. Matthews, et al. (2004). “Electrospinning collagen and elastin: preliminary vascular tissue engineering.” Frontiers in Bioscience 9: 1422-1432.
Bunting, S., L. D. Silvio, et al. (2005). “Bioresorbable glass fibres facilitate peripheral nerve regeneration.” Journal of Hand Surgery (British and European Volume) 30B(3): 242-247.
Cai, J., X. Peng, et al. (2004). “Synergistic improvements in cell and axonal migration across sciatic nerve lesion gaps using bioresorbable filaments and heregulin-b1.” J Biomed Mater Res 69A: 247-258.
Ceballos, D., X. Navarro, et al. (1999). “Magnetically aligned collagen gel filling a collagen nerve guide improves peripheral nerve regeneration.” Experimental Neurology 158: 290-300.
Ceballos, D., A. Valero-cabre, et al. (2002). “Morphologic and functional evaluation of peripheral nerve fibers regenerated through polyimide sieve electrodes over long-term implantation.” J Biomed Mater Res 60: 517-528.
Chen, R. R. and D. J. Mooney (2003). “Polymeric growth factor delivery strategies for tissue engineering.” Pharmaceutical Research 20(8): 1103-1112.
Chew, S. Y., T. C. Hufnagel, et al. “Mechanical properties of single electrospun drug-encapsulated nanofibres.” Nanotechnology (Submitted).
Chew, S. Y., J. Wen, et al. (2005). “Sustained release of proteins from electrospun biodegradable fibers.” Biomacromolecules 6(4): 2017-2024.
Dahiyat, B. I., M. Richards, and K. W. Leong, Controlled release from poly(phosphoester) matrices. Journal of Controlled Release, 1995. 33: p. 13-21.
Dahlin, L. B. and G. Lundborg (1999). “Bridging defects in nerve continuity: influence of variations in synthetic fiber composition.” Journal of Materials Science: Materials in Medicine 10: 549-553.
Fansa, H., T. Dodic, et al. (2003). “Tissue engineering of peripheral nerves: epineural grafts with application of cultured Schwann cells.” Microsurgery 23: 72-77.
Francel, P. C., K. S. Smith, et al. (2003). “Regeneration of rat sciatic nerve across a LactoSorb bioresorbable conduit with interposed short-segment nerve grafts.” J. Neurosurg 99(549-554).
Fridrikh, S. V., J. H. Yu, M. P. Brenner, and G. C. Rutledge, Controlling the Fiber Diameter during Electrospinning. Physical Review Letters, 2003. 90(14): p. 144502-1-144502-4.
Frostick, S. P., Q. Yin, et al. (1998). “Schwann cells, neurotrophic factors and peripheral nerve regeneration.” Microsurgery 18: 397-405.
Greene, L. A. and A. S. Tischler, Establishment of a noradrenergic clonal line of rat adrenal pheochromocytoma cells which respond to nerve growth factor. Pro Natl Acad Sci U.S.A, 1976. 73(7): p. 2424-2428.
Green, L. A., Nerve growth factor prevents the death and stimulates the neuronal differentiation of clonal PC12 pheochromocytoma cells in serum-free medium. Journal of Cell Biology, 1978. 78: p. 747-755.
Heine, W., K. Conant, et al. (2004). “Transplanted neural stem cells promote axonal regeneration through chronically denervated peripheral nerves” Experimental Neurology 189(2): 231-240.
Hoke, A., T. Ho, et al. (2003). “Glial cell line-derived neurotrophic factor alters axon Schwann cell units and promotes myelination in unmyelinated nerve fibers.” The Journal of Neuroscience 23(2): 561-567.
Huang, Z. M., Y. Z. Zhang, M. Kotaki, and S. Ramakrishna, A review on polymer nanofibers by electrospinning and their applications in nanocomposites. Composites science and technology, 2003. 63(15): p. 2223-2253.
Huang, L., K. Nagapudi, et al. (2001). “Engineered collagen-PEO nanofibers and fabrics.” Journal of biomaterial science, Polymer Edition 12(9): 979-993.
Huatan, H., J. H. Collett, et al. (1995). “Preparation and characterization of poly(e-caprolactone) polymer blends for the delivery of proteins.” Biomaterials 16: 1297-1303.
Huatan, H., J. H. Collett, D. Attwood, and C. Booth, Preparation and characterization of poly(e-caprolactone) polymer blends for the delivery of proteins. Biomaterials, 1995. 16: p. 1297-1303.
Jiang, H., D. Fang, et al. (2004). “Preparation and characterization of ibuprofen-loaded poly(lactide-co-clycolide)/poly(ethylene glycol)-g-chitosan electrospun membranes.” J. Biomater. Sci. Polymer Edn. 15(3): 279-296.
Jin, H.-J., J. Chen, et al. (2004). “Human bone marrow stromal cell responses to electrospun silk fibroin mats.” Biomaterials 25: 1039-1047.
Johansson, F., P. Carlberg, et al. (2005). “Axonal outgrowth on nano-imprinted patterns.” Biomaterials 27(8): 1251-1258.
Katzir, I., J. Shani, K. Regev, D. Shabashov, and P. Lazarovici, A quantitative bioassay for nerve growth factor, using PC12 clones expressing different levels of trkA receptors. J Mol Neurosci, 2002. 18(3): p. 251-64.
Kenawy, E.-R., G. L. Bowlin, et al. (2002). “Release of tetracycline hydrochloride from electrospun poly(ethylene-co-vinylacetate, poly(lactic acid), and a blend.” Journal of Controlled Release 81: 57-64
Kim, K., Y. K. Luu, et al. (2004). “Incorporation and controlled release of a hydrophilic antibiotic using poly(lactide-co-glycolide)-based electrospun nanofibrous scaffolds.” Journal of Controlled Release 98: 47-56.
Li, W. J., C. T. Laurencin, E. J. Caterson, R. S. Tuan, and F. K. Ko, Electrospun nanofibrous structure: a novel scaffold for tissue engineering. Journal of biomedical material research, 2002. 60(4): p. 613-621. Li, D. and Y. Xia, Direct fabrication of composite and ceramic hollow nanofibers by electrospinning. Nano Letters, 2004. 4(5): p. 933-938.
Luu, Y. K., K. Kim, B. S. Hsiao, B. Chu, and M. Hadjiargyrou, Development of a nanostructured DNA delivery scaffold via electrospinning of PLGA and PLA-PEG block copolymers. Journal of Controlled Release, 2003. 89: p. 341-353.
Matthews, J. A., E. D. Boland, et al. (2003). “Electrospinning of collagen type II: a feasibility study.” Journal of bioactive and compatible polymers 18: 125-134.
Matthews, J. A., G. E. Wnek, et al. (2002). “Electrospinning of collagen nanofibers.” Biomacromolecules 3(2): 232-238.
Mao, H. Q., I. Kadiyala, Z. Zhao, W. Dang, and K. Leong, Biodegradable polyphosphoesters, in Encyclopedia of Controlled Drug Delivery, M. E., Editor. 1999, Johns Wiley & Sons: New York, N.Y.
Medlicott, N. J., I. G. Tucker, et al. (1996). “Chlorhexidine release from poly(e-caprolactone) films prepared by solvent evaporation.” International Journal of Pharmaceutics 143: 25-35.
Mimura, T., M. Dezawa, et al. (2004). “Peripheral nerve regeneration by transplantation of bone marrow stromal cell-derived Schwann cells in adult rats.” J Neurosurg 101: 806-812.
Min, B. M., G. Lee, et al. (2004). “Electrospinning of silk fibroin nanofibers and its effect on the adhesion and spreading of normal human keratinocytes and fibroblasts in vitro.” Biomaterials 25(7-8): 1289-1297.
Ngo, T.-T. B., P. J. Waggoner, et al. (2003). “Poly(L-Lactide) microfilaments enhance peripheral nerve regeneration across extended nerve lesions.” Journal of Neuroscience Research 72: 227-238.
Pean, J. M., M. C. Venier-Julienne, F. Boury, and P. Menei, NGF release from poly(D,L-lactide-co-glycolide) microspheres. Effect of some formulation parameters on encapsulated NGF stability. Journal of Controlled Release, 1998. 56: p. 175-187.
Rangappa, N., A. Romero, et al. (2000). “Laminin-coated poly(L-lactide) filaments induce robust neurite growth while providing directional orientation.” J Biomed Mater Res 51: 625-634.
Reneker, D. H. and I. Chun, Nanometre diameter fibres of polymer, produced by electrospinning. Nanotechnology, 1996. 7: p. 216-223.
Ritger, P. L. and N. A. Peppas, A simple equation for description of solute release I. Fickian and non-Fickian release from non-swellable devices in the forms of slabs, spheres, cylinders or discs. Journal of Controlled Release, 1987. 5(1): p. 23-36.
Rosner, B. I., R. A. Siegel, et al. (2003). “Rational design of contact guiding, neurotrophic matrices for peripheral nerve regeneration.” Annals of Biomedical Engineering 31: 1383-1401.
Rutkowski, G. E., C. A. Miller, et al. (2004). “Synergistic effects of micropattemed biodegradable conduits and Schwann cells on sciatic nerve regeneration.” J. Neural. Eng. 1: 151-157.
Sanders, E. H., R. Kloefkorn, et al. (2003). “Two-phase electrospinning from a single electrified jet: microencapsulation of aqueous reservoirs in poly(ethylene-co-vinyl acetate) fibers.” Macromolecules 36(11): 3803-3805.
Schmalenberg, K. E. and K. E. Uhrich (2005). “Micropattemed polymer substrates control alignment of proliferating Schwann cells to direct neuronal regeneration.” Biomaterials 26: 1423-1430.
Shingo, T., I. Date, et al. (2002). “Neuroprotective and restorative effects of intrastriatal grafting of encapsulated GDNF-producing cells in a rat model of Parkinson's disease.” Journal of Neuroscience Research 69: 946-954.
Stang, F., H. Fansa, et al. (2005). “Structural parameters of collagen nerve grafts influence peripheral nerve regeneration.” Biomaterials 26: 3083-3091.
Stitzel, J. D., K. J. Pawlowski, et al. (2001). “Arterial smooth muscle cell proliferation on a novel biomimicking, biodegradable vascular graft scaffold.” Journal of biomaterials applications 16: 22-33.
Sun, Z., E. Zussman, A. L. Yarin, J. H. Wendorff, and A. Greiner, Compound core-shell polymer nanofibers by co-electrospinning. Advanced materials, 2003. 15(22): p. 1929-1932.
Terenghi, G. (1999). “Peripheral nerve regeneration and neurotrophic factors.” J. Anat. 194: 1-14.
Thoenen, H. and Y. A. Barde, Physiology of nerve growth factor. Physiol Rev, 1980. 60(4): p. 1284-335.
Thompson, D. M. and H. M. Beuttner (2004). “Oriented Schwann cell monolayers for directed neurite outgrowth.” Annals of Biomedical Engineering 32(8): 1120-1130.
Tornqvist, N., L. Bjorklund, et al. (2000). “Implantation of bioactive growth factor-secreting rods enhances fetal dopaminergic graft survival, outgrowth density and functional recovery in a rat model of Parkinson's disease.” Experimental Neurology 164: 130-138.
Tria, M. A., M. Fusco, G. Vantini, and R. Mariot, Pharmacokinetics of nerve growth factor (NGF) following different routes of administration to adult rats. Experimental Neurology, 1994. 127: p. 178-183.
Udina, E., F. J. Rodriguez, et al. (2004). “FK506 enhances regeneration of axons across long peripheral nerve gaps repaired with collagen guides seeded with allogeneic Schwann cells.” GLIA 47: 120-129.
Wang, S., Q. Cai, et al. (2002). “Acceleration effect of basic fibroblast growth factor on the regeneration of peripheral nerve through a 15-mm gap.” J Biomed Mater Res 66A: 522-531.
Wen, J. and R. X. Zhuo (1998). “Preparation and characterization of poly(D,L-lactide-coethylene methyl phosphate).” Polymer International 47(4): 503-509.
Wen, J. and R. X. Zhuo, Preparation and characterization of poly(D,L-lactide-coethylene methyl phosphate). Polymer International, 1998. 47(4): p. 503-509.
Wen, J., G. J. A. Kim, and K. W. Leong, Poly(lactide-co-ethyl ethylene phosphate)s as new drug carriers. Journal of Controlled Release, 2003. 92(1-2): p. 39-48.
Wnek, G. E., M. E. Carr, et al. (2003). “Electrospinning of Nanofiber Fibrinogen Structures.” Nano Letters 3(2): 213-216.
Yoshii, S., M. Shima, et al. (2004). “Nerve regeneration along collagen filament and the presence of distal nerve stump.” Neurological Research 26: 145-150.
Yoshimoto, H., Y. M. Shin, et al. (2003). “A biodegradable nanofiber scaffold by electrospinning and its potential for bone tissue engineering.” Biomaterials 24: 2077-2082.
Zeng, J., X. Xu, et al. (2003). “Biodegradable electrospun fibers for drug delivery.” Journal of Controlled Release 92: 227-231.
Zhang, Y., Z.-M. Huang, X. Xu, C. T. Lim, and S. Ramakrishna, Preparation of core-shell structured PCL-r-gelatin bi-component nanofibers by coaxial electrospinning. Chemistry of Materials, 2004. 16: p. 3406-3409.
Zong, X., K. Kim, D. Fang, S. Ran, B. S. Hsiao, and B. Chu, Structure and process relationship of electrospun bioadsorbable nanofiber membrane. Polymer, 2002. 43: p. 4403-4412.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An electrospun fiber composition comprising one or more polymers and one or more biological therapeutics; or
an electrospun fiber composition comprising PCLEEP and one or more therapeutically active molecules; or
a cylindrical polymer film comprising an inner surface, an outer surface, and a lumen, wherein an electrospun fiber composition comprising one or more polymers and one or more therapeutically active molecules is present on the inner or outer surface of the cylindrical polymer film.

2. The electrospun fiber composition of claim 1, wherein the one or more biological therapeutics are selected from the group consisting of a polypeptide, polypeptide fragment, nucleic acid molecule, and carbohydrates.

3. The electrospun fiber composition of claim 2, wherein the one or more biological therapeutics comprise a polypeptide.

4. The electrospun fiber composition of claim 3, wherein the polypeptide is a growth factor, chemokine, cytokine, receptor, antibody, scFv, antibody fragment or combinations thereof.

5. The electrospun fiber composition of claim 3, further comprising an additional polypeptide.

6. The electrospun fiber composition of claim 5, wherein the additional polypeptide is a filler polypeptide.

7. The electrospun fiber composition of claim 6, wherein the filler polypeptide is human serum albumin.

8. The electrospun fiber composition of claim 1, wherein the electrospun fibers are randomly oriented fibers.

9. The electrospun fiber composition of claim 1, wherein the electrospun fibers are aligned fibers.

10. The electrospun fiber composition of claim 1, wherein the electrospun fiber is produced by uniaxial electrospinning.

11. The electrospun fiber composition of claim 1, wherein the electrospun fiber is produced by coaxial electrospinning.

12. The electrospun fiber composition of claim 1, wherein the electrospun fiber is produced by multiaxial electrospinning.

13. The electrospun fiber composition of claim 1, wherein the average fiber diameter is between about 10 nm and 10 um.

14. The electrospun fiber composition of claim 12, wherein the average fiber diameter is between about 100 um and 1 um.

15. The electrospun fiber composition of claim 1, wherein the one or more polymers comprise a synthetic polymer, a natural polymer, a protein engineered biopolymer or a combination thereof.

16. The electrospun fiber composition of claim 15, wherein the one or more polymers comprise a polyester or derivative thereof.

17. The electrospun fiber composition of claim 16, wherein the polyester is a poly(phosphoester) polymer.

18. The electrospun fiber composition of claim 16, wherein the polyester is poly (ε-caprolactore-co-ethyl ethylene phosphate (PCLEEP).

19.-61. (canceled)

62. A nerve guide conduit comprising a cylindrical polymer film comprising on the interior surface an electrospun fiber composition comprising one or more polymers and one or more therapeutically active molecules that induce nerve growth.

63.-70. (canceled)

71. A polymer film comprising on one or both surfaces an electrospun fiber composition comprising one or more polymers and one or more biologically active molecules.

72.-97. (canceled)
